(19) United States
(12) Patent Application Publication
(10) Pub. No.: US 2016/0058387 A1
(43) Pub. Date: Mar. 3, 2016

(54) IMAGING APPARATUS AND CONTROL METHOD THEREFOR, AND LIGHTING SYSTEM AND CONTROL METHOD THEREFOR

(71) Applicant: CANON KABUSHIKI KAISHA, Tokyo (JP)

(72) Inventor: Kiwamu Kobayashi, Kawasaki-shi (JP)

(21) Appl. No.: 14/839,681

(22) Filed: Aug. 28, 2015

(30) Foreign Application Priority Data
Sep. 2, 2014 (JP) 2014-178481
Sep. 2, 2014 (JP) 2014-178483

(51) Int. Cl. A61B 5/00; A61B 5/11

(52) U.S. Cl. CPC A61B 5/7214; A61B 5/0071; A61B 5/1128; A61B 5/7278

(57) ABSTRACT

The disclosure provides at least one imaging apparatus, at least one lighting system, one or more methods of controlling same and one or more storage mediums. In one or more embodiments of the at least one imaging apparatus, the at least one lighting system, control methods therefor and storage mediums, an identification unit configured to identify a timing in an extinction period of a first illumination light at or during which an operator who performs a work on an object does not recognize an extinction of the first illumination light, and an image pickup unit configured to perform imaging of the object at the timing in the extinction period identified by the identification unit to pick up the object image, are provided.

### BACKGROUND OF THE INVENTION

1. Field of the Invention

The present disclosure relates to at least one imaging apparatus configured to perform imaging of an object, at least one control method for the imaging apparatus, at least one lighting system configured to illuminate an object with illumination light, at least one control method for the lighting system, and a storage medium storing a program for causing a computer to execute the at least one control method.

2. Description of the Related Art

In recent years, research and practical application of pathological imaging with regard to visualization and imaging of a lesion by some form of methods have been carried out.

For example, in fluorescence imaging or the like, it is becoming possible to visually check the presence or absence of tumor cells without a large scale apparatus mainly in the field of animal experiments. Japanese Patent No. 3482440 and Japanese Patent Laid-Open No. 2006-180926 disclose recent developments in this field.

In medical practices too, not only a so-called biopsy diagnosis in related art but also a pathology examination may be performed in mid-course of a surgical operation in recent years. For example, in an operation for resecting the tumor cells, an examination such as X-ray CT, MRI, or PET is performed in advance, and the operation is prepared by previously identifying a resection range.

However, in actuality, various determinations and alternations related to an operation strategy are made depending on a situation of a resected part during the operation. At this time, a pathological diagnosis of the resected part may be urgently needed in some cases, and an intraoperative rapid pathological diagnosis (intraoperative rapid cell diagnosis) is actually conducted.

In the current intraoperative pathological diagnosis, a part corresponding to an observation objective is partially resected, and the resected part is visualized by a technique such as fluorescence imaging under a predetermined different environment, so that the urgent pathological diagnosis is carried out on the basis of this visualized part.

As described above, the intraoperative pathology examination is needed to carry out the appropriate operation. An environment where the pathological imaging related to the pathology examination or the like can be conducted on site during the operation without interrupting the operational procedure is preferably created.

The following advantages are attained when the pathological imaging and the diagnosis can be conducted in an actual time on site during the operation.
It is not necessary to perform tissue resection to prepare a sample of the pathology examination. That is, damage to human tissues caused by the examination is avoided.
Since a resected cut area can be observed, it is possible to appropriately resect a lesion such as tumor. That is, a likelihood of left-over of lesion tissues such as tumor cells is low, and also, decrease in the quality of life of a patient caused by excessively wide resection can be avoided.
Since the operation can be conducted while pathological information is obtained, the operation can be carried out by changing the strategy in response to the situation in a flexible and impromptu manner, and it is possible to provide the optimal operation to the patient.
Since the operation is not interrupted by waiting for the pathology examination and its result, a time for the patient to be at laparotomy can be minimized, and it is possible to minimize the patient burden.
Since the surgical work is not disturbed, a risk of the operation itself can be suppressed to minimum.

With the above-described advantages, it is important to make it possible to perform the pathological imaging without interrupting the various works on site during the operation.

However, in general, since the pathological imaging for the pathology examination represented by the fluorescence imaging or the like involves a low luminance, the pathological imaging is performed in an environment where the external light is excluded. Hereinafter, imaging that requires such an environment is referred to as “low luminance imaging”.

According to Japanese Patent No. 3482440 described above, it is described that the fluorescence imaging is premised on the environment where the external light is excluded.

On the other hand, the illumination of an operation room is set to be bright on its nature, and in particular, the illumination of an operative field is set to be extremely bright. Whereas the brightness of a sick bay is normally 100 lx to 200 lx, the brightness of the operation room is in the region of 1,000 lx, and the brightness of the operative field is in the region of 20,000 lx.

With regard to an objective of intraoperative pathological imaging, the operative field is mainly the object, but as described above, it is difficult to perform the low luminance imaging under the bright illumination even when an optical filter is used. In addition, the optical filter may not be effective in some cases depending on a relationship between a wavelength to be imaged and a wavelength of the external light.

On the other hand, in a case where the pathological imaging is prioritized, an option for adopting a method of temporarily turning off the illumination of the operation room as described in Japanese Patent Laid-Open No. 2006-180926 exists. However, the various works are interrupted, or the patient is put into a risky situation in darkness, so that this method is not also considered as appropriate.

Furthermore, in a case where the low luminance imaging is performed, it is necessary to increase the amount of light exposure to a needed level while the external light is excluded.

To achieve this, a method with which the exposure time is extended, a diaphragm is opened, the sensitivity of an image pickup element is improved, or the like is also conceivable. In addition, in the case of the fluorescence imaging, a method of increasing the intensity of excited light instead of flash illumination light is conceivable.

However, for example, in a case where the fluorescence imaging is performed during the operation, it is difficult to perform the low luminance imaging of the moving object when faced with one or more of the following aspects or condition(s).
The object corresponding to the objective of the fluorescence imaging is not a sample but a human body, and the object is the human cells as a treatment objective. Therefore, it is necessary to suppress the damage to the tissues caused by the excitation to minimum. That is, the intensity of the excited light needs to be suppressed to necessity minimum. In other words, the level of the excited light is not allowed to be immoderately increased to gain the exposure amount in the imaging.
Since a part of the living patient corresponds to the object, the object is somewhat moved because of various involuntary motions such as pulsating, aspiration, and peristaltic motion, and the like. Furthermore, the object is deformed or moved because of the works of the operation and the procedure which are performed in a simultaneous parallel manner too. In addition, during the operation, the imaging apparatus may not be necessarily firmly fixed. Therefore, it is difficult to perform the exposure for a long time in the imaging under the above-described conditions.

With the above-described condition(s), it is difficult to perform the low luminance imaging of the moving object. That is, up to now, it is difficult to obtain an appropriate object image of the moving object while the works are conducted on this moving object.

### SUMMARY OF THE INVENTION

The present disclosure provides at least one system in which an appropriate object image can be obtained without disturbing works of an operator who performs the works on an object, such as a moving object.

At least one imaging apparatus that performs imaging of an object in an environment irradiated with illumination light by a lighting system according to an aspect of the present disclosure includes: an input unit configured to input an instruction for performing the imaging; an identification unit configured to identify a timing in an extinction period of the illumination light at or during which an operator who performs a work on the object does not recognize an extinction of the illumination light corresponding to a timing after the instruction for performing the imaging is input from the input unit; and an image pickup unit configured to perform the imaging of the object to pick up an object image at the timing in the extinction period identified by the identification unit.

At least one lighting system that is communicable with an imaging apparatus configured to perform imaging of an object and irradiates the object with illumination light having a first light intensity according to another aspect of the present disclosure includes: a communication unit configured to perform a communication with the imaging apparatus and receive extinction instruction information related to a timing in an extinction period of the illumination light from the imaging apparatus; and a control unit configured to control an extinction of the illumination light on a basis of the extinction instruction information and perform a control to emit illumination light having a second light intensity higher than the first light intensity in at least one of a period immediately before and a period immediately after the extinction period of the illumination light on a basis of a predetermined rule. Furthermore, an imaging apparatus according to another aspect of the present disclosure includes: an image pickup unit configured to perform imaging of an object and obtain an image; a timing control unit configured to control a timing related to a first imaging performed by the image pickup unit in a state in which the object is irradiated with a first illumination light and a timing related to a second imaging performed by the image pickup unit in a state in which the object is irradiated with a second illumination light that is different from the first illumination light or a state in which the object is irradiated with none of the first illumination light and the second illumination light; a first obtaining unit configured to obtain a plurality of first images by performing the first imaging a plurality of times by the image pickup unit; a second obtaining unit configured to obtain a plurality of second images by performing the second imaging a plurality of times by the image pickup unit; a detection unit configured to detect motion information related to a motion of the object between the plurality of the first images obtained by the first obtaining unit; and a generation unit configured to perform a first processing on the plurality of the second images by the second obtaining unit on a basis of the motion information detected by the detection unit and perform a second processing on the plurality of the second images on which the first processing has been performed to generate one output image.

According to other aspects of the present disclosure, one or more additional imaging apparatuses, one or more additional imaging systems, one or more control methods therefor, and one or more storage mediums are discussed herein. Further features of the present disclosure will become apparent from the following description of exemplary embodiments with reference to the attached drawings.

### BRIEF DESCRIPTION OF THE DRAWINGS

FIG. 1 illustrates an example outline configuration of an imaging system according to first, fifth, and sixth exemplary embodiments.

FIGS. 2A and 2B illustrate example internal configurations of a lighting system and an imaging apparatus illustrated in FIG. 1 according to the first, fifth, and sixth exemplary embodiments.

FIG. 3 is a timing chart illustrating an example processing procedure of a control method by the imaging system illustrated in FIG. 1 according to the first exemplary embodiment.

FIG. 4 illustrates examples of a control waveform related to light intensity in instantaneous extinction and a time change of brightness perceived by a human being according to the first exemplary embodiment.

FIG. 5 illustrates an example of instantaneous extinction imaging based on single exposure according to the first exemplary embodiment.

FIG. 6 illustrates an example of instantaneous extinction imaging based on plural exposures according to the first exemplary embodiment.

FIG. 7 illustrates an example the instantaneous extinction imaging based on the single exposure according to a second exemplary embodiment.

FIG. 8 illustrates an example of the instantaneous extinction imaging based on the plural exposures according to the second exemplary embodiment.

FIG. 9 illustrates an example outline configuration of an imaging system according to a third exemplary embodiment.

FIGS. 10A and 10B illustrate example internal configurations of a lighting system and an imaging apparatus illustrated in FIG. 9.

FIGS. 11A to 11E illustrate examples of a typical light receiving waveform of first illumination light received by a light receiving unit of the imaging apparatus illustrated in FIG. 9 and FIG. 10B according to the third exemplary embodiment.

FIG. 12 is a flow chart illustrating an example of a processing procedure by an imaging system according to a fourth exemplary embodiment.

FIG. 13 is a timing chart illustrating an example processing procedure of the control method by the imaging system illustrated in FIG. 1 according to the fifth exemplary embodiment.

FIG. 14 is a timing chart illustrating examples of a light emission timing of the first illumination light in a normal light emission period and a periodic extinction period and examples of a light emission timing and an exposure timing of second illumination light in the periodic extinction period according to the fifth exemplary embodiment.

FIG. 15 is an explanatory diagram for describing motion information detecting processing by the imaging apparatus according to the fifth exemplary embodiment.

FIG. 16 is an explanatory diagram for describing the motion information detecting processing by the imaging apparatus according to the fifth exemplary embodiment.

FIG. 17 is an explanatory diagram for describing the motion information detecting processing by the imaging apparatus according to the fifth exemplary embodiment.

FIG. 18 is a timing chart illustrating an example of a light emission timing of the first illumination light, and examples of a light emission timing of the second illumination light, an exposure timing, and an image output timing according to the sixth exemplary embodiment.

FIG. 19 illustrates an example functional configuration of the imaging apparatus according to the sixth exemplary embodiment.

FIG. 20 is an explanatory diagram for describing calculation processing by using respective frame memories by a control and processing unit illustrated in FIG. 19 according to the sixth exemplary embodiment.

FIG. 21 is an explanatory diagram for describing the calculation processing by using the respective frame memories by the control and processing unit illustrated in FIG. 19 according to the sixth exemplary embodiment.

### DESCRIPTION OF THE EMBODIMENTS

Hereinafter, with reference to the drawings, exemplary embodiments of the present disclosure will be described.

### First Exemplary Embodiment

First, a first exemplary embodiment of the present disclosure will be described.

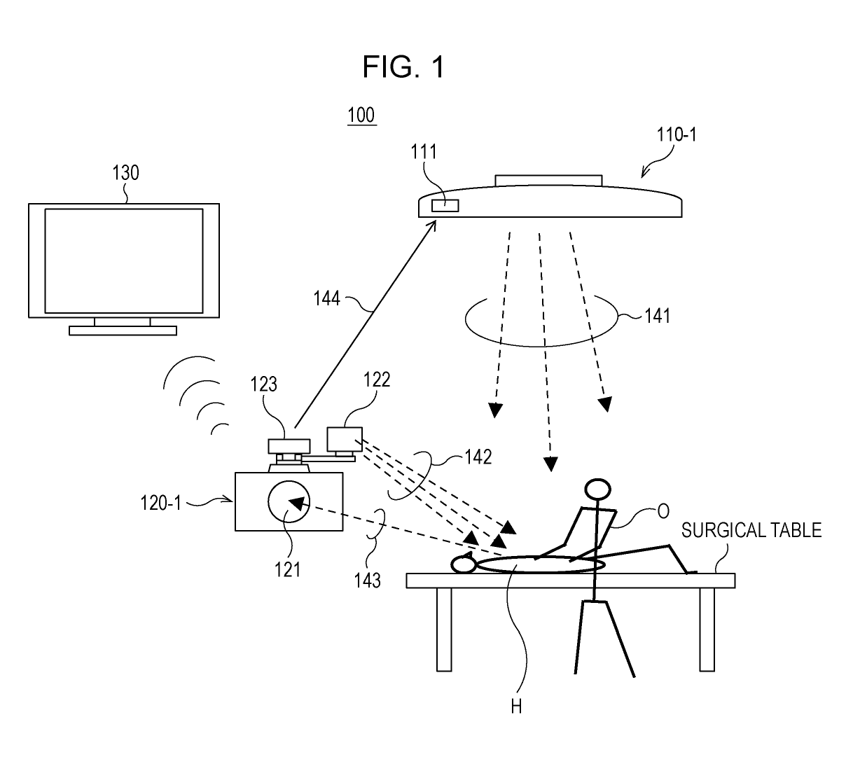

FIG. 1 illustrates an example outline configuration of an imaging system 100 according to the first exemplary embodiment of the present disclosure. It is noted that, according to the present exemplary embodiment, when a surgeon corresponding to an operator O performs a surgical work on a patient laid on a surgical table in the operation room, a case is supposed where the imaging apparatus 120-1 performs the low luminance imaging while this patient corresponds to an object H.

As illustrated in FIG. 1, the imaging system 100 is constituted by including a lighting system 110-1, the imaging apparatus 120-1, and a display apparatus 130.

The lighting system 110-1 is constituted so as to be communicable with the imaging apparatus 120-1 that performs the imaging of the object H and irradiates the object H with illumination light (first illumination light) 141. Herein, the lighting system 110-1 is set to irradiate the object H with the first illumination light 141 having a first light intensity in a normal case. The luminance of the first illumination light 141 is considerably higher than normal illumination light, and the luminance of the light is particularly high with respect to the operative field. In addition, the first illumination light 141 is light that satisfies conditions of shadowless light which are necessary for the operation room.

The lighting system 110-1 is also provided with a communication unit 111 configured to perform a communication with the imaging apparatus 120-1 and receive extinction instruction information related to a timing in an extinction period of the first illumination light 141 (including information of the extinction period of the first illumination light 141) and the like from the imaging apparatus 120-1.

The imaging apparatus 120-1 performs the imaging of the object H in the environment irradiated with the first illumination light 141 by the lighting system 110-1. Specifically, the imaging apparatus 120-1 according to the present exemplary embodiment identifies a timing at which the operator O who performs the work on the object H does not recognize the extinction of the first illumination light 141 in the extinction period of the first illumination light 141 which corresponds to a timing after the instruction for performing the imaging is input and performs the imaging of the object H at this extinction timing to pick up an object image. As the imaging apparatus 120-1, a shape like a general single-lens reflex camera or a compact camera is supposed, but the configuration does not necessarily need to be the above-described shape. In addition, for example, the imaging apparatus 120-1 can perform the fluorescence imaging.

The imaging apparatus 120-1 is constituted by including an image pickup unit 121 that includes an image pickup optical system such as a lens and a diaphragm and an image pickup element, a light emitting unit 122, and a communication unit 123.

The image pickup unit 121 performs the imaging of the object H to pick up an object image based on light 143 from the object H.

The light emitting unit 122 emits second illumination light 142 that is different from the first illumination light 141 in accordance with a purpose of this imaging to the object H for exposure when the imaging of the object H is performed by the image pickup unit 121. For example, in a case where the purpose of the imaging is the fluorescence imaging, the second illumination light 142 is light for exciting a predetermined fluorescent material. In a case where the purpose of the imaging is normal flash imaging, for example, the second illumination light 142 is flash light.

The communication unit 123 performs a communication with the lighting system 110-1 and the display apparatus 130. The communication unit 123 transmits, for example, a wireless signal (infrared signal) 144 such as the extinction instruction information related to the timing in the extinction period of the first illumination light 141 (including the information of the extinction period of the first illumination light 141) to the lighting system 110-1. The communication unit 123 also transmits, for example, the object image picked up by the image pickup unit 121 and the like to the display apparatus 130.

The display apparatus 130 performs a communication with the imaging apparatus 120-1 (the communication unit 123) and performs processing of receiving the object image picked up by the image pickup unit 121 and the like and displaying this object image and the like.

Next, internal configurations of the lighting system 110-1 and the imaging apparatus 120-1 illustrated in FIG. 1 will be described.

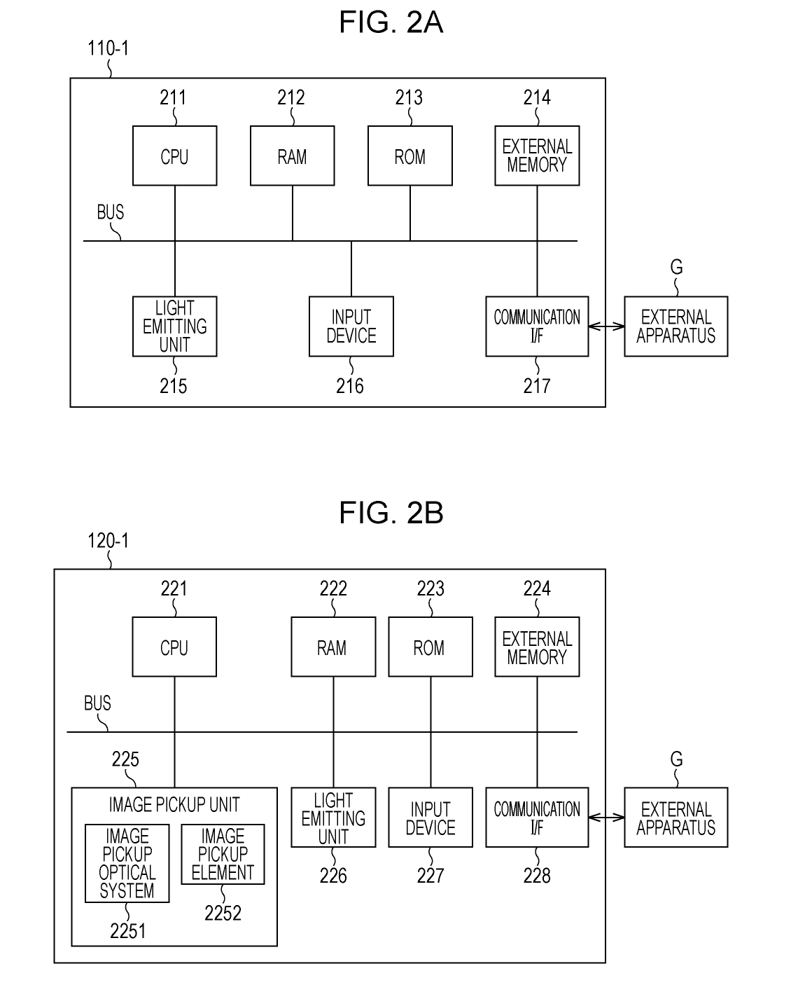

FIGS. 2A and 2B illustrate examples of the internal configurations of the lighting system 110-1 and the imaging apparatus 120-1 illustrated in FIG. 1. Specifically, FIG. 2A illustrates the example of the internal configuration of the lighting system 110-1 illustrated in FIG. 1, and FIG. 2B illustrates the example of the internal configuration of the imaging apparatus 120-1 illustrated in FIG. 1.

As illustrated in FIG. 2A, the lighting system 110-1 is constituted by including a CPU 211, a RAM 212, the ROM 213, an external memory 214, a light emitting unit 215, an input device 216, and a communication interface (communication I/F) 217. The respective configurations illustrated in FIG. 2A are constituted to be mutually communicable via a bus.

The CPU 211 controls the operation of the lighting system 110-1 in an overall manner by using, for example, the program or data stored in the ROM 213 or the external memory 214.

The RAM 212 is provided with an area for temporarily storing the program or data loaded from the ROM 213 or the external memory 214 and is also provided with a work area used for the CPU 211 to perform the various processings.

The ROM 213 stores the program in which no change is needed, the information such as various parameters, and the like.

The external memory 214 stores, for example, an operating system (OS) and the program executed by the CPU 211 and further stores the information already given in the descriptions in the present exemplary embodiment and the like. It is noted that, according to the exemplary embodiment, the external memory 214 stores the program for executing the processing according to the exemplary embodiment of the present disclosure, but for example, a mode in which the program is stored in the ROM 213 may also be applied to one or more embodiments of the present disclosure.

The light emitting unit 215 emits the first illumination light 141 on the basis of the control by the CPU 211.

The input device 216 is constituted, for example, by a switch, a button, or the like (including a power supply switch) installed in the lighting system 110-1.

The communication I/F 217 governs transmission and reception of various information and the like which are performed between the lighting system 110-1 and an external apparatus G (in the present example, the imaging apparatus 120-1).

Herein, the communication unit 111 illustrated in FIG. 1 is constituted from the communication I/F 217 illustrated in FIG. 2A.

As illustrated in FIG. 2B, the imaging apparatus 120-1 is constituted by including the CPU 221, a RAM 222, a ROM 223, an external memory 224, an image pickup unit 225, a light emitting unit 226, an input device 227, and a communication interface (communication I/F) 228. The respective configurations illustrated in FIG. 2B are constituted to be mutually communicable via a bus.

The CPU 221 controls the operation of the imaging apparatus 120-1 in an overall manner by using, for example, the program or data stored in the ROM 223 or the external memory 224.

The RAM 222 is provided with an area for temporarily storing the program or data loaded from the ROM 223 or the external memory 224 and is also provided with a work area used for the CPU 221 to perform the various processings.

The ROM 223 stores the program in which no change is needed, the information such as various parameters, and the like.

The external memory 224 stores, for example, an operating system (OS) and the program executed by the CPU 221 and further stores the information already given in the descriptions in the present exemplary embodiment and the like. It is noted that, according to the exemplary embodiment, the program for executing the processing according to the exemplary embodiment of the present invention is stored in the external memory 224, but for example, a mode in which the program is stored in the ROM 223 may also be applied to one or more embodiments of the present disclosure.

The image pickup unit 225 performs the imaging of the object H to pick up an object image based on the light 143 from the object H. Specifically, the image pickup unit 225 is constituted by including an image pickup optical system 2251 such as a lens for guiding the light 143 from the object H to an internal image pickup element 2252 and the diaphragm and the image pickup element 2252 that picks up the object image based on the light 143 from the object H guided via the image pickup optical system 2251.

The light emitting unit 226 emits the second illumination light 142 on the basis of the control by the CPU 221.

The input device 227 is constituted, for example, by a switch, a button, or the like installed in the imaging apparatus 120-1. The input device 227 is used, for example, by the user to perform various instructions to the imaging apparatus 120-1 to input the instructions to the CPU 221 or the like.

The communication I/F 228 governs transmission and reception of various information and the like which are performed between the imaging apparatus 120-1 and the external apparatus G (in the present example, the lighting system 110-1 and the display apparatus 130).

Herein, the image pickup unit 121 illustrated in FIG. 1 is constituted from the image pickup unit 225 illustrated in FIG. 2B. The light emitting unit 122 illustrated in FIG. 1 is constituted from the light emitting unit 226 illustrated in FIG. 2B. The communication unit 123 illustrated in FIG. 1 is constituted from the communication I/F 228 illustrated in FIG. 2B.

Hereinafter, the exposure performed on the basis of the first illumination light 141 is referred to as first exposure, and the exposure performed on the basis of the second illumination light 142 is referred to as second exposure.

According to the present exemplary embodiment, the low luminance imaging based on the second illumination light 142 or the like is performed without disturbance with respect to the illumination by the first illumination light 141 for supporting the surgical work by the surgeon corresponding to the operator O and also without disturbance by the first illumination light 141.

Herein, the disturbance with respect to the illumination by the first illumination light 141 refers to a state in which the work is even momentarily interrupted because extinction occurs in a time period during which the operator O under this illumination can recognize the extinction, or flickering occurs to a level at which the operator O is bothered and loses concentration.

The disturbance by the first illumination light 141 refers to a state in which the first illumination light 141 becomes disturbance noise when the imaging of the low luminance imaging or the like is performed, and the intended imaging of the low luminance imaging or the like is not performed.

According to the present exemplary embodiment, the illumination by the first illumination light 141 is turned off in only an extinction period corresponding to a time period during which the operator O as a human being does not recognize the extinction, and the imaging such as the low luminance imaging is performed in the extinction period. At this time, in the case of the first illumination light 141 that turns on and off at a predetermined period (that turns off at least in a time related to the above-described extinction period), the imaging such as the low luminance imaging is performed in the extinction period at a gap between the light emissions of the first illumination light 141.

In addition, according to the present exemplary embodiment, the lighting system 110-1 is supposed to be a device that can perform high-speed response. Up to now, the lighting system of this type is often a halogen lamp or the like and is not necessarily a device that can perform the high-speed response. However, in recent years, an LED and organic electroluminescence are put to practical use as illumination devices, the lighting system that uses these devices can perform the high-speed response. In general, reasons for demanding these devices are mainly high light emission efficiency (that is, low heat generation), high luminance, long life, wide wavelength characteristic options, and the like, but attention is paid on the high-speed responsiveness according to the present exemplary embodiment.

Next, a processing procedure of a control method by the imaging system 100 illustrated in FIG. 1 will be described.

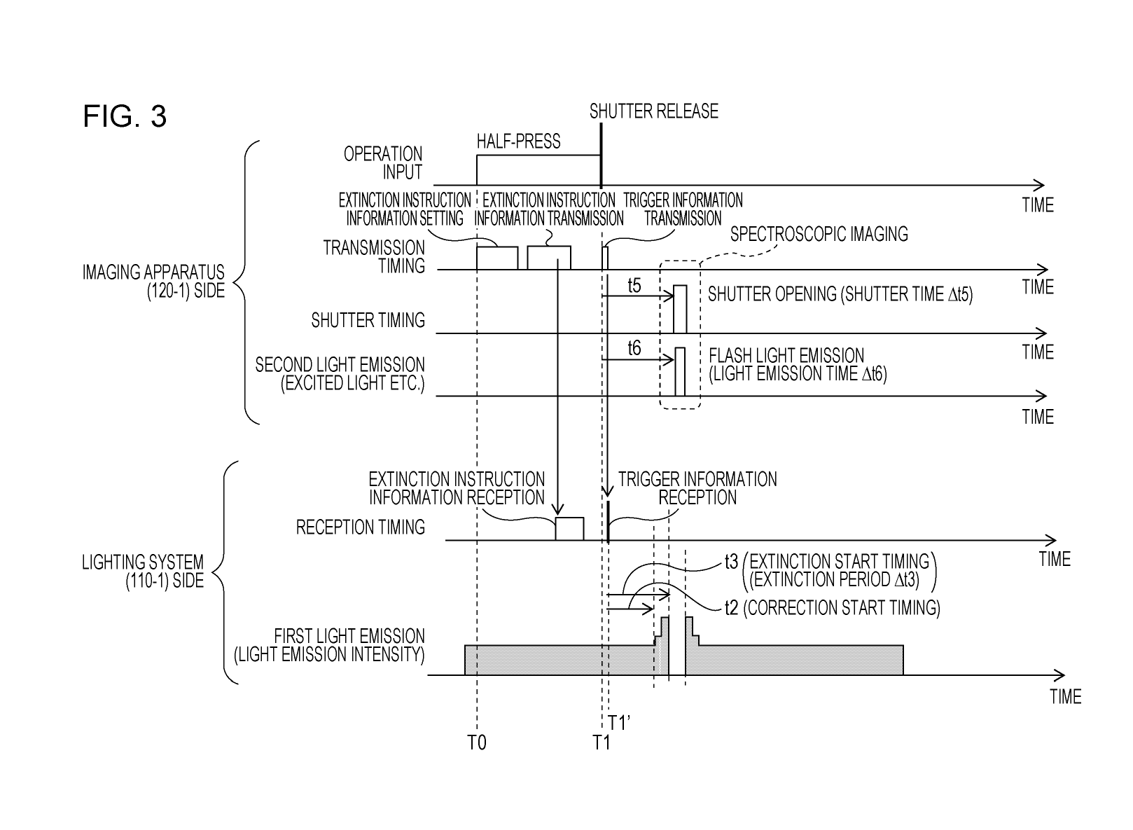

FIG. 3 is a timing chart illustrating an example processing procedure of the control method by the imaging system 100 illustrated in FIG. 1.

In FIG. 3, the first to fourth stages from the top correspond to sequences of the imaging apparatus 120-1, and the fifth and sixth stages from the top correspond to sequences of the lighting system 110-1.

The sequence on the first stage from the top in FIG. 3 indicates the operation input from the user via the input device 227 of the imaging apparatus 120-1. In the example illustrated in FIG. 3, half-press of a shutter button of the input device 227 is started at a time T0, and indentation of the shutter button of the input device 227 is performed at a time T1. Herein, the input device 227 constitutes an input unit configured to input the instruction for performing the imaging.

The sequence on the second stage from the top in FIG. 3 indicates the transmission timing by the communication I/F 228 of the imaging apparatus 120-1 (the communication unit 123) to the lighting system 110-1.

First, when the half-press of the shutter button of the input device 227 occurs at the time T0, the CPU 221 of the imaging apparatus 120-1 identifies a timing at which the operator O who performs the work on the object H does not recognize the extinction of the first illumination light 141 in the extinction period of the first illumination light 141 from a surrounding situation or the like. The CPU 221 that performs the processing of identifying the timing in the extinction period of the first illumination light 141 constitutes an identification unit.

Subsequently, the CPU 221 of the imaging apparatus 120-1 sets the extinction instruction information related to the identified timing in the extinction period (including the information of the extinction period). Specifically, in the example illustrated in FIG. 3, the extinction instruction information indicating that the first illumination light 141 is turned off is set at a timing in an extinction period Δt3 after an elapse of a time t3 since the trigger information indicating that the indentation of the shutter button has been performed is received by the lighting system 110-1. Thereafter, the communication I/F 228 of the imaging apparatus 120-1 (the communication unit 123) transmits the extinction instruction information to the lighting system 110-1.

When the indentation of the shutter button of the input device 227 is performed, the CPU 221 of the imaging apparatus 120-1 detects this state. Then, the communication I/F 228 of the imaging apparatus 120-1 (the communication unit 123) transmits the trigger information indicating that the indentation of the shutter button has been performed to the lighting system 110-1.

The sequence on the third stage from the top in FIG. 3 indicates that an electronic shutter is opened (exposure is started) after an elapse of a time t5 since the trigger information is generated. Specifically, the CPU 221 of the imaging apparatus 120-1 sets a shutter time corresponding to a time during which the shutter is opened in accordance with the extinction period of the first illumination light 141 instructed with respect to the lighting system 110-1. The CPU 221 that performs the processing of setting this shutter time constitutes a setting unit.

The sequence on the fourth stage from the top in FIG. 3 indicates that light emission of the second illumination light 142 (second light emission) is performed from the light emitting unit 226 after an elapse of a time t6 since the trigger information is generated.

The sequence on the fifth stage from the top in FIG. 3 indicates a reception timing by the communication I/F 217 of the lighting system 110-1 (the communication unit 111). Specifically, in the example illustrated in FIG. 3, the reception timings of the extinction instruction information and the trigger information transmitted from the imaging apparatus 120-1 are indicated.

The sequence on the sixth stage from the top in FIG. 3 indicates illumination/extinction timings of the first illumination light 141 by the light emitting unit 215 of the lighting system 110-1 and emission light intensity thereof. Specifically, while a trigger timing T1′ set while the communication I/F 217 (the communication unit 111) receives the trigger information is used as a reference, the CPU 211 of the lighting system 110-1 performs light emission intensity correction after an elapse of a time t2 and also performs extinction in the extinction period Δt3 after an elapse of the time t3. It is noted that, according to the exemplary embodiment, t1≈t1′ is set to simplify the descriptions. In the example illustrated in FIG. 3, the CPU 211 of the lighting system 110-1 performs a control to emit the illumination light having a second light intensity that is higher than a first light intensity corresponding to a normal light intensity of the first illumination light 141 in both periods including a period immediately before and a period immediately after the extinction period Δt3 of the first illumination light 141 on the basis of a predetermined rule as the light emission intensity correction. It is noted that, in the example illustrated in FIG. 3, the correction for increasing the light intensity in both the periods including the period immediately before and the period immediately after the extinction period Δt3 is performed, but the present disclosure is not limited to this configuration. For example, a mode in which the correction for increasing the light intensity in at least one of the periods including the period immediately before the extinction period Δt3 and the period immediately after the extinction period Δt3 is performed is also included in the present disclosure.

In the case of the example illustrated in FIG. 3, the CPU 221 of the imaging apparatus 120-1 sets a shutter time Δt5 equivalent to an exposure period in the period of the extinction period Δt3 and also sets a light emission period Δt6 as a substantially same timing as the shutter time Δt5. Then, the image pickup unit 225 of the imaging apparatus 120-1 performs the imaging of the object H on the basis of the control by the CPU 221 at the timing in the extinction period of the first illumination light 141 to pick up the object image.

Next, a visual characteristic of a human being in the extinction period Δt3 of the instantaneous extinction illustrated in FIG. 3 will be described.

Herein, according to the present exemplary embodiment, the extinction period Δt3 of the instantaneous extinction illustrated in FIG. 3 is suppressed to such an extent that the instantaneous extinction is not recognized as flickering or the like within a range of human visual responsiveness. Specifically, the extinction period Δt3 of FIG. 3 corresponding to the instantaneous extinction is suppressed to such an extent that the operator O does not recognize the extinction of the first illumination light 141.

For this reason, an upper limit is set for the length of the extinction period Δt3, and also the light emission intensity correction for increasing the light intensity in at least one of the period immediately before the instantaneous extinction and the period immediately after the instantaneous extinction is performed.

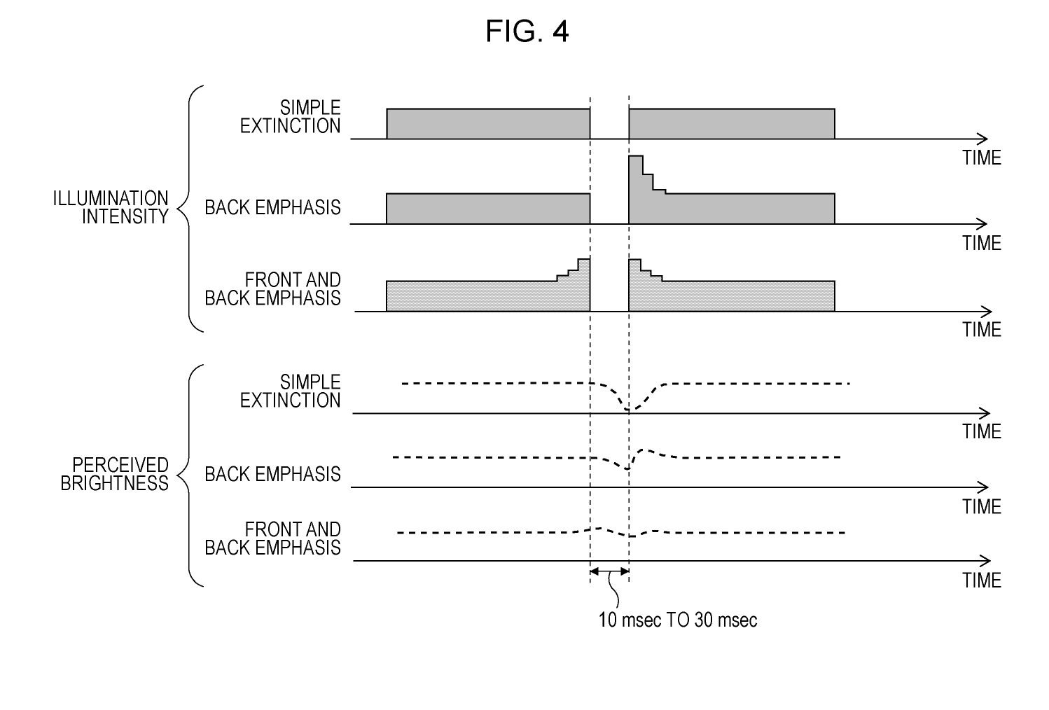

FIG. 4 illustrates examples of a control waveform related to the light intensity in the instantaneous extinction and a time change of brightness perceived by the human being according to the first exemplary embodiment of the present disclosure.

The first to third stages from the top in FIG. 4 indicate a case of simple extinction where extinction is simply performed, a case of back emphasis where the light intensity is increased only immediately after the extinction, and a case of front and back emphasis where the light intensities are increased in both periods immediately before the extinction and immediately after the extinction in the stated order from the top.

In general, human eyes have an integration characteristic in a time direction, and this can be represented as Expression (1) below.

B(T)=∫_{t=T}^{t=T+τ}gamma(L(T))dt (1)
τ=20 msec˜50 msec

In Expression (1), B(T) denotes a brightness perceived at a time T. L(T) denotes a light intensity of the illumination light (the first illumination light 141) at the time T. A function gamma ( ) denotes a perceptual (visual) sensitivity characteristic with respect to the light intensity. τ denotes a constant of the integration and is 20 msec to 50 msec although relatively large differences exist between individuals.

The fourth to sixth stages from the top in FIG. 4 respectively indicate changes of values obtained by multiplying the control waveform related to the light intensity in the instantaneous extinction on the first to third stages from the top in FIG. 4 by the time integration characteristic indicated by Expression (1) (the time integration characteristic obtained by correcting the light intensity of the illumination light on the basis of the visual sensitivity characteristic).

As illustrated in FIG. 4, it is possible to reduce the extent of variations of the perceived brightness by performing the back emphasis or the front and back emphasis as compared with the case of the simple extinction. In the case of the same level of the brightness variation, it is possible to relatively set the extinction period to be longer.

According to the present exemplary embodiment, the front and back maximum emphasis amount is set as 200%, and the extinction period Δt3 is set as 10 msec to 30 msec.

As illustrated in FIG. 4 and Expression (1), the CPU 211 of the lighting system 110-1 performs the light emission intensity correction (the front and back emphasis, the back emphasis, or the like) on the basis of a predetermined rule in which a change ratio in the extinction period Δt3 of the value obtained by correcting the light intensity of the illumination light by the time integration characteristic based on the visual sensitivity characteristic becomes lower than that of the illumination light that keeps the first light intensity as it is (simple extinction of FIG. 4).

Next, a case where the plural exposures are performed will be described.

In the descriptions by using FIG. 3 and FIG. 4, the first illumination light 141 emitted from the lighting system 110-1 is supposed to be the light that has the predetermined light intensity with respect to the passage of time.

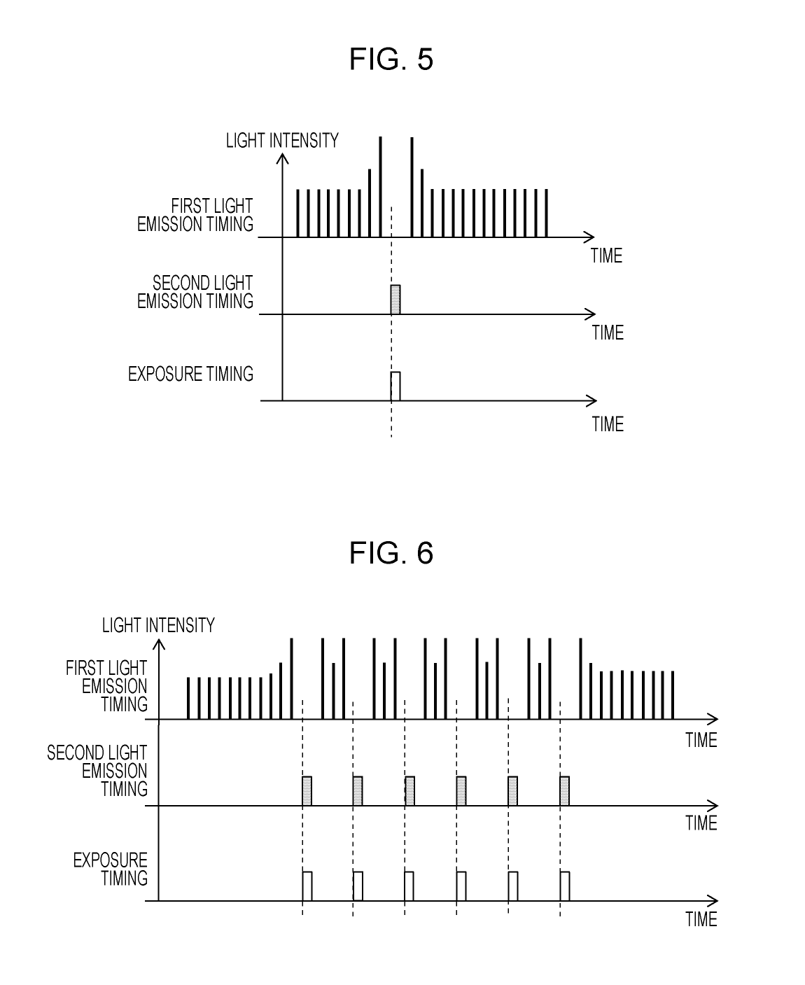

On the other hand, a large number of LED illuminations and organic electroluminescence illuminations in actuality perform driving in an impulse manner. In view of the above, from FIG. 5 and subsequent drawings, an example in which the light that turns on by the impulse driving is supposed as the first illumination light 141 will be described.

The frequency of the first illumination light 141 in the case of the impulse driving is approximately several hundreds of hertz to 1 kilohertz. It is noted that a pulse train of FIG. 5 and subsequent drawings is schematically represented, and a pitch of the pulse train does not correctly represent a lighting frequency.

FIG. 5 illustrates an example of instantaneous extinction imaging based on the single exposure according to the first exemplary embodiment of the present disclosure. FIG. 6 illustrates an example of instantaneous extinction imaging based on the plural exposures according to the first exemplary embodiment of the present disclosure.

In FIG. 5 and FIG. 6, the first stage from the top indicates a first light emission timing by the first illumination light 141, the second stage from the top indicates a second light emission timing by the second illumination light 142, and the third stage from the top indicates an exposure timing by the image pickup unit 225.

For example, in the case of the low luminance imaging, some exposure time is still needed even with backing-up by improvement in the sensitivity of the image pickup element 2252, release of the diaphragm corresponding to one of components of the image pickup optical system 2251, and the like. However, according to the present exemplary embodiment, since the extinction period Δt3 has an upper limit to avoid the flickering or the like, and the single exposure time is not sufficiently extended.

In this case, as illustrated in FIG. 6, the plural instantaneous extinctions and the plural exposures are carried out to obtain the sufficient exposure amount.

As illustrated in FIG. 6, even when the instantaneous extinction imaging is performed plural times, upper limits of the respective extinction times are observed, and the predetermined correction emphasis on the light intensity is performed in the periods before and after the respective extinction periods and the like. With this configuration, as a result of the calculation of Expression (1) with respect to the waveform of the first illumination light 141 like the first stage of FIG. 6, the respective instantaneous extinctions can be suppressed to such an extent that the state is not recognized as the flickering or the like.

According to the present exemplary embodiment, it is possible to obtain the appropriate object image without disturbing the operator O (surgeon) who performs the work (surgery) on the object H. Accordingly, for example, the pathological diagnosis or the like can be performed in the actual time on site on the basis of the object image during the surgery, and it is possible to carry out the appropriate surgery.

### Second Exemplary Embodiment

Next, a second exemplary embodiment of the present disclosure will be described.

The second exemplary embodiment corresponds to a mode in which the light emitting unit 122 of the imaging apparatus 120-1 illustrated in FIG. 1 (the light emitting unit 226 of the imaging apparatus 120-1 illustrated in FIG. 2B) is not needed.

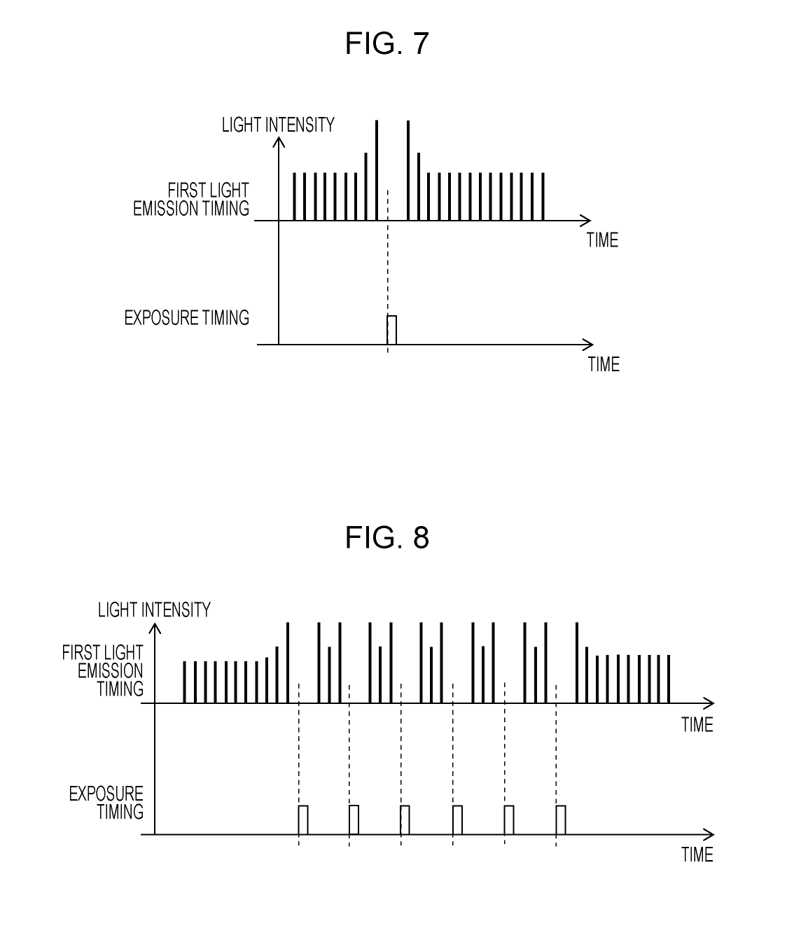

FIG. 7 illustrates an example of the instantaneous extinction imaging based on the single exposure according to the second exemplary embodiment of the present disclosure. Specifically, the configuration of FIG. 7 is obtained by deleting the second light emission timing from FIG. 5.

FIG. 8 illustrates an example of the instantaneous extinction imaging based on the plural exposures according to the second exemplary embodiment of the present disclosure. Specifically, the configuration of FIG. 8 is obtained by deleting the second light emission timing from FIG. 6.

In FIG. 7 and FIG. 8, the first stage from the top indicates the first light emission timing by the first illumination light 141, and the second stage from the top indicates the exposure timing by the image pickup unit 225.

As described above, the second exemplary embodiment corresponds to a mode in which the second light emission (for example, the excited light in the fluorescence imaging) is not needed in the second exposure. That is, the second exemplary embodiment corresponds to the mode for performing the imaging on the basis of a light emission principle in which light emission occurs by itself by way of some type of energy.

The second exemplary embodiment has a similar configuration and a similar operation to those of the first exemplary embodiment other than no provision of the light emitting unit in the imaging apparatus 120-1.

### Third Exemplary Embodiment

Next, a third exemplary embodiment of the present disclosure will be described.

The third exemplary embodiment is devised to propose conditions for performing the low luminance imaging or the like when the instantaneous extinction occurs in the first light emission by the first illumination light 141.

According to the first and second exemplary embodiments described above, the extinction instruction information related to the timing in the extinction period Δt3 of the instantaneous extinction is transmitted from the imaging apparatus 120-1 to the lighting system 110-1 to instruct the instantaneous extinction.

On the other hand, in a case where the first light emission by the first illumination light 141 is impulse light emission, a non-illumination timing at a gap (gap) between the impulse light emissions can be used as the preexistent instantaneous extinction (timing in the extinction period Δt3) in some cases. According to the third exemplary embodiment, this is utilized.

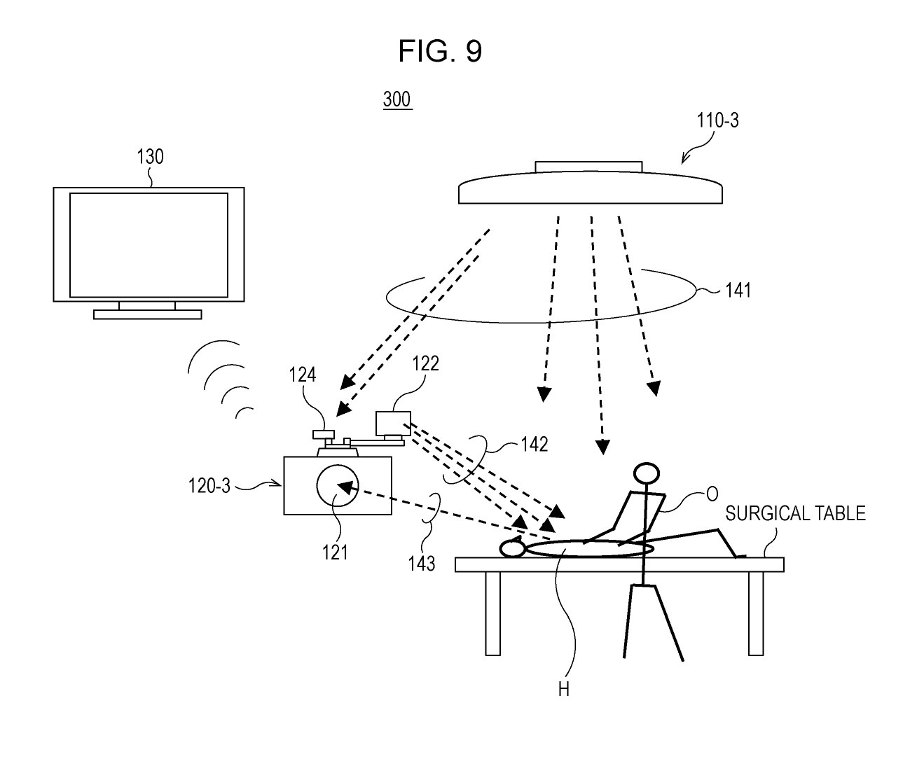

FIG. 9 illustrates an example schematic configuration an imaging system 300 according to the third exemplary embodiment of the present disclosure. Herein, in FIG. 9, configurations similar to the configurations illustrated in FIG. 1 are assigned with the same reference symbols, and descriptions thereof will be omitted.

As illustrated in FIG. 9, the imaging system 300 is constituted by including a lighting system 110-3, an imaging apparatus 120-3, and the display apparatus 130.

Differences from the imaging system 100 illustrated in FIG. 1 according to the first exemplary embodiment include a configuration in which the communication unit 111 is removed from the lighting system 110-3 and a configuration in which the communication unit 123 is removed from the imaging apparatus 120-3, and also a light receiving unit 124 is newly provided in the imaging apparatus 120-3.

The light receiving unit 124 is configured to receive the first illumination light 141.

Next, internal configurations of the lighting system 110-3 and the imaging apparatus 120-3 illustrated in FIG. 9 will be described.

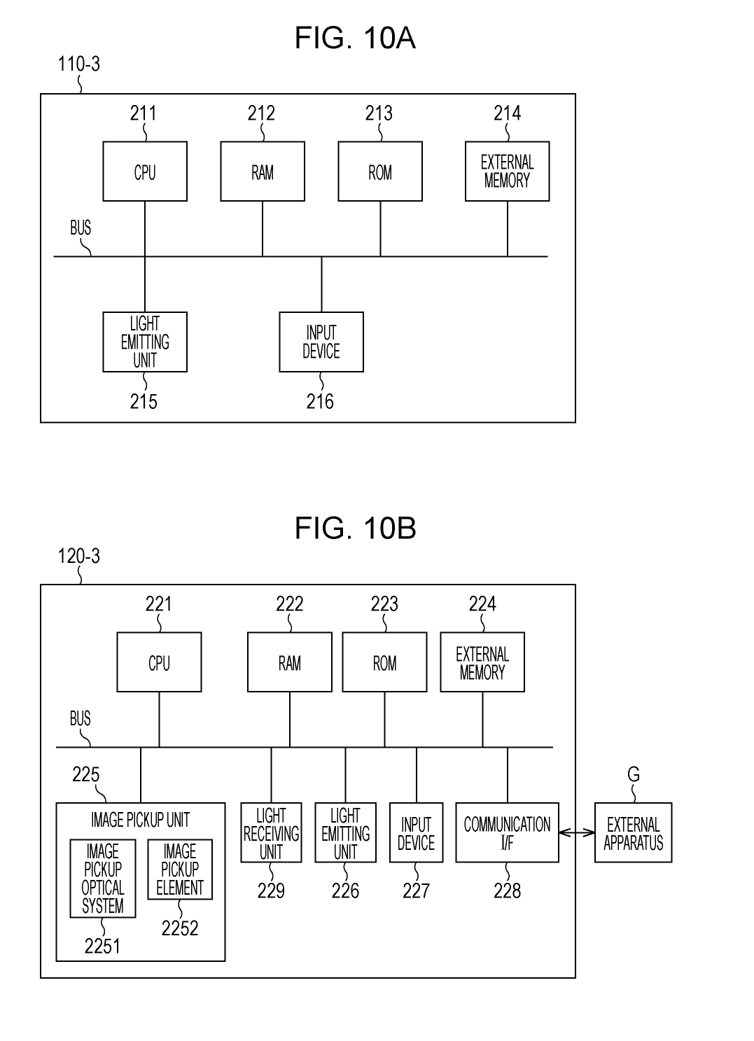

FIGS. 10A and 10B illustrate example internal configurations of the lighting system 110-3 and the imaging apparatus 120-3 illustrated in FIG. 9. Specifically, FIG. 10A illustrates the example internal configuration of the lighting system 110-3 illustrated in FIG. 9, and FIG. 10B illustrates the example internal configuration of the imaging apparatus 120-3 illustrated in FIG. 9. Herein, in FIGS. 10A and 10B, configurations similar to the configurations illustrated in FIGS. 2A and 2B are assigned the same reference symbols, and descriptions thereof will be omitted.

As illustrated in FIG. 10A, the lighting system 110-3 is constituted by including the CPU 211, the RAM 212, the ROM 213, the external memory 214, the light emitting unit 215, and the input device 216. Specifically, the lighting system 110-3 is by removing the communication interface (communication I/F) 217 from the configuration of the lighting system 110-1 illustrated in FIG. 2A.

As illustrated in FIG. 10B, the imaging apparatus 120-3 is constituted by including the CPU 221, the RAM 222, the ROM 223, the external memory 224, the image pickup unit 225, the light emitting unit 226, the input device 227, the communication I/F 228, and a light receiving unit 229.

At this time, the communication I/F 228 governs transmission and reception of various information and the like which are performed between the imaging apparatus 120-3 and the display apparatus 130 corresponding to the external apparatus G. That is, the exemplary embodiment is different from the first exemplary embodiment and the like in that the communication I/F 228 does not perform a communication with the lighting system 110-3 corresponding to an external apparatus.

The light receiving unit 229 is configured to receive the first illumination light 141. The light receiving unit 124 illustrated in FIG. 9 is constituted from the light receiving unit 229 illustrated in FIG. 10B.

According to the present exemplary embodiment, the first illumination light 141 is received by the light receiving unit 229 of the imaging apparatus 120-3 (the light receiving unit 124), and the CPU 221 of the imaging apparatus 120-3 evaluates the waveform of the first illumination light 141 received by the light receiving unit 229. Then, the CPU 221 of the imaging apparatus 120-3 determines whether or not an imaging timing can be set in a gap between the illuminations of the first illumination light 141 in accordance with this evaluation result. Then, in a case where the imaging timing can be set in the gap between the illuminations of the first illumination light 141, the CPU 221 of the imaging apparatus 120-3 sets the gap imaging timing (at this time, for example, the timing in the extinction period Δt3 of the first illumination light 141 is identified). Then, the image pickup unit 225 of the imaging apparatus 120-3 performs the exposure in accordance with the set gap imaging timing and the imaging of the object image of the object H on the basis of the control by the CPU 221 of the imaging apparatus 120-3.

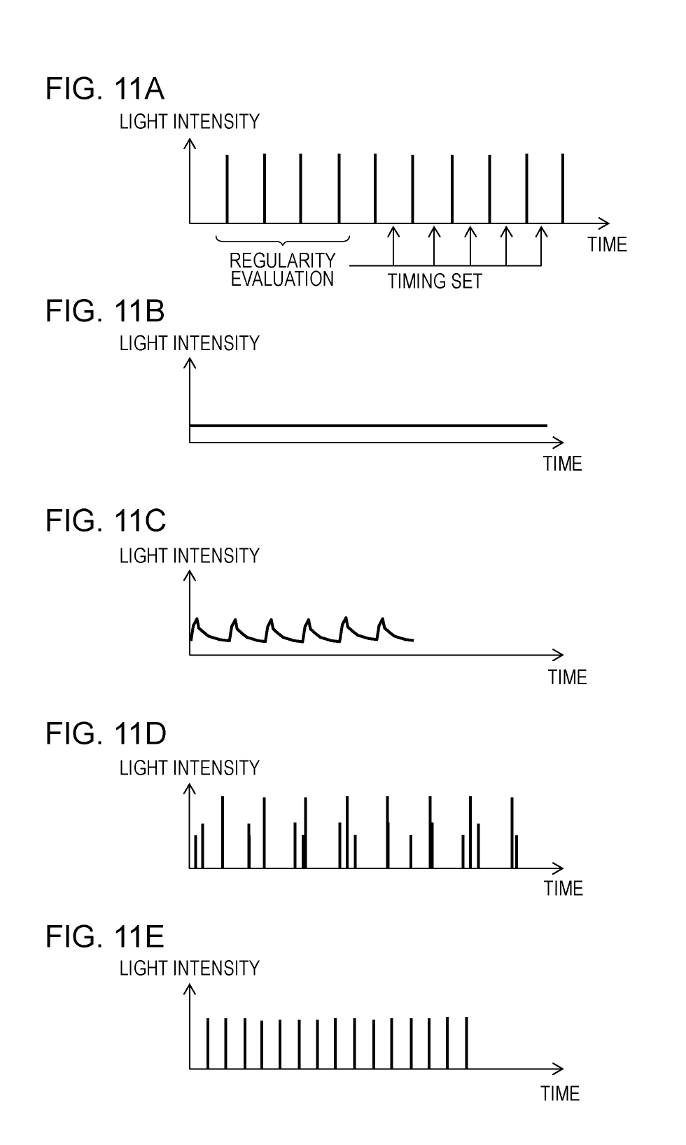

FIGS. 11A to 11E illustrate examples of a typical light receiving waveform of the first illumination light 141 received by the light receiving unit 124 of the imaging apparatus 120-3 illustrated in FIG. 9 (the light receiving unit 229 of the imaging apparatus 120-3 illustrated in FIG. 10B) according to the third exemplary embodiment of the present disclosure. Hereinafter, whether or not the gap imaging timing can be set with regard to each of the light receiving waveforms of the first illumination light 141 will be described.

A lighting pattern 1 illustrated in FIG. 11A corresponds to a case where existences of periodicity of the light receiving waveform of the first illumination light 141 and non-light emission time are clear, and also a length of the non-light emission time (gap time between light emissions) is long with respect to an operation speed of the imaging apparatus 120-3. In this case, since the CPU 221 of the imaging apparatus 120-3 can perform the exposure once or more within the non-light emission time, the setting of the gap imaging timing is possible, and it is determined that the gap imaging can be performed. Furthermore, the CPU 221 of the imaging apparatus 120-3 sets an exposure timing on the basis of the determination that the gap imaging can be performed. Herein, since the exposure time for each exposure is short, in a case where it is necessary to perform the plural exposures, a plurality of exposure timings are set as illustrated in FIG. 11A.

A lighting pattern 2 illustrated in FIG. 11B corresponds to a case where the first illumination light 141 is not the impulse light emission but is continuous light emission. In this case, it is not possible for the CPU 221 of the imaging apparatus 120-3 to set the gap imaging timing, and it is determined that the gap imaging is not performed.

A lighting pattern 3 illustrated in FIG. 11C corresponds to a case where afterglow is so large that the non-light emission period does not occur even when the first illumination light 141 is the impulse light emission. In this case, it is not possible for the CPU 221 of the imaging apparatus 120-3 to set the gap imaging timing, and it is determined that the gap imaging is not performed.

A lighting pattern 4 illustrated in FIG. 11D corresponds to a case where a plurality of lighting patterns in which the light receiving waveforms of the first illumination light 141 have different periods are overlapped with each other. In this case, it is not possible for the CPU 221 of the imaging apparatus 120-3 to set the gap imaging timing except for a case where the plurality of periods have a particular relationship, and it is determined that the gap imaging is not performed.

A lighting pattern 5 illustrated in FIG. 11E corresponds to a case where the light receiving waveform of the first illumination light 141 has periodicity, and also even when the non-light emission period exists, this non-light emission period has a length in which the single exposure is not to be performed. In this case, it is not possible for the CPU 221 of the imaging apparatus 120-3 to set the gap imaging timing, and it is determined that the gap imaging is not performed.

### Fourth Exemplary Embodiment

Next, a fourth exemplary embodiment of the present disclosure will be described.

A schematic configuration of the imaging system according to the fourth exemplary embodiment is obtained by combining the lighting system 110-1 illustrated in FIG. 2A with the imaging apparatus 120-3 illustrated in FIG. 10B. At this time, the communication I/F 228 of the imaging apparatus 120-3 illustrated in FIG. 10B communicates with the lighting system 110-3 corresponding to the external apparatus G.

When the imaging system according to the fourth exemplary embodiment adopts the above-described configuration, the following processing can be realized. That is, only when it is determined that it is not possible to perform the gap imaging according to the third exemplary embodiment, the processing according to the first exemplary embodiment is executed.

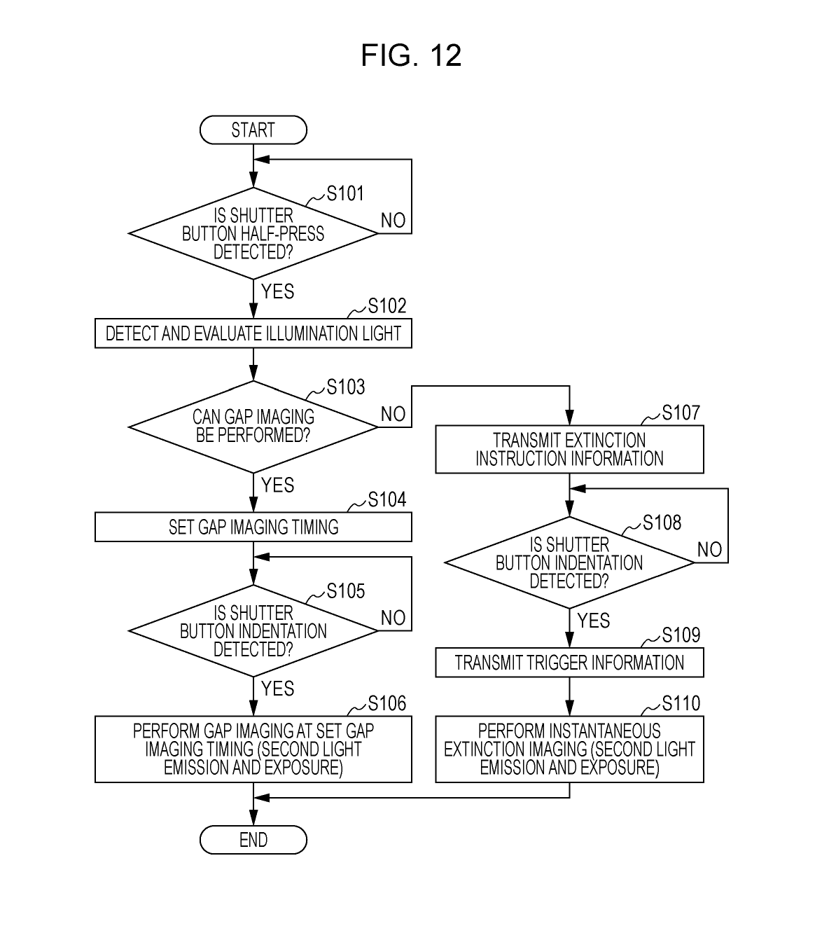

FIG. 12 is a flow chart illustrating an example processing procedure of a control method by an imaging system according to the fourth exemplary embodiment of the present disclosure.

First, in step S101, the CPU 221 of the imaging apparatus 120-3 determines whether or not the half-press of the shutter button of the input device 227 is detected. As a result of this determination, in a case where the half-press of the shutter button of the input device 227 is not detected (S101/NO), until the half-press of the shutter button of the input device 227 is detected, the flow stands by in step S101.

On the other hand, as a result of the determination in step S101, in a case where the half-press of the shutter button of the input device 227 is detected (S101/YES), the flow advances to step S102.

When the flow advances to step S102, once the light receiving unit 229 of the imaging apparatus 120-3 receives the first illumination light 141, the CPU 221 of the imaging apparatus 120-3 detects this reception and evaluates the first illumination light 141. The contents of this evaluation are as described in the third exemplary embodiment.

Subsequently, in step S103, the CPU 221 of the imaging apparatus 120-3 determines whether or not the gap imaging described in the third exemplary embodiment can be performed on the basis of the result of the evaluation in step S102.

As a result of the determination in step S103, in a case where the gap imaging can be performed (S103/YES), the flow advances to step S104.

When the flow advances to step S104, the CPU 221 of the imaging apparatus 120-3 performs the setting of the gap imaging timing described according to the third exemplary embodiment.

Subsequently, in step S105, the CPU 221 of the imaging apparatus 120-3 determines whether or not the indentation of the shutter button of the input device 227 is detected. As a result of this determination, in a case where the indentation of the shutter button of the input device 227 is not detected (S105/NO), until the indentation of the shutter button of the input device 227 is detected, the process stands by in step S105.

On the other hand, as a result of the determination in step S105, in a case where the indentation of the shutter button of the input device 227 is detected, (S105/YES), the flow advances to step S106.

When the flow advances to step S106, the light emitting unit 226 and the image pickup unit 225 of the imaging apparatus 120-3 performs the gap imaging (the second light emission and exposure) of the object H on the basis of the control by the CPU 221 of the imaging apparatus 120-3 at the gap imaging timing set in step S104.

When the processing in step S106 is ended, the processing of the flow chart in FIG. 12 is ended.

As a result of the determination in step S103, in a case where it is not possible to perform the gap imaging (S103/NO), the flow advances to step S107.

When the flow advances to step S107, the CPU 221 of the imaging apparatus 120-3 performs the control to transmit the extinction instruction information described in the first exemplary embodiment to the lighting system 110-1 via the communication I/F 228.

Subsequently, in step S108, the CPU 221 of the imaging apparatus 120-3 determines whether or not the indentation of the shutter button of the input device 227 is detected. As a result of this determination, in a case where the indentation of the shutter button of the input device 227 is not detected (S108/NO), until the indentation of the shutter button of the input device 227 is detected, the process stands by in step S108.

On the other hand, as a result of the determination in step S108, in a case where the indentation of the shutter button of the input device 227 is detected, (S108/YES), the flow advances to step S109.

When the flow advances to step S109, the CPU 221 of the imaging apparatus 120-3 performs the control to transmit the trigger information described in the first exemplary embodiment to the lighting system 110-1 via the communication I/F 228.

Subsequently, in step S110, the light emitting unit 226 and the image pickup unit 225 of the imaging apparatus 120-3 performs the instantaneous extinction imaging (the second light emission and exposure) described in the first exemplary embodiment on the object H on the basis of the control by the CPU 221 of the imaging apparatus 120-3.

When the processing in step S110 is ended, the processing of the flow chart in FIG. 12 is ended.

### Fifth Exemplary Embodiment

First, a fifth exemplary embodiment of the present disclosure will be described.

A configuration of the present exemplary embodiment is the same as the configuration of the first exemplary embodiment illustrated in FIG. 1 and FIGS. 2A and 2B.

Although the configuration of the present exemplary embodiment is the same as that of the first exemplary embodiment, operation timings of the imaging apparatus 120-1 and the lighting system 110-1 are different.

A processing procedure of a control method by the imaging system 100 of FIG. 1 according to the present exemplary embodiment will be described.

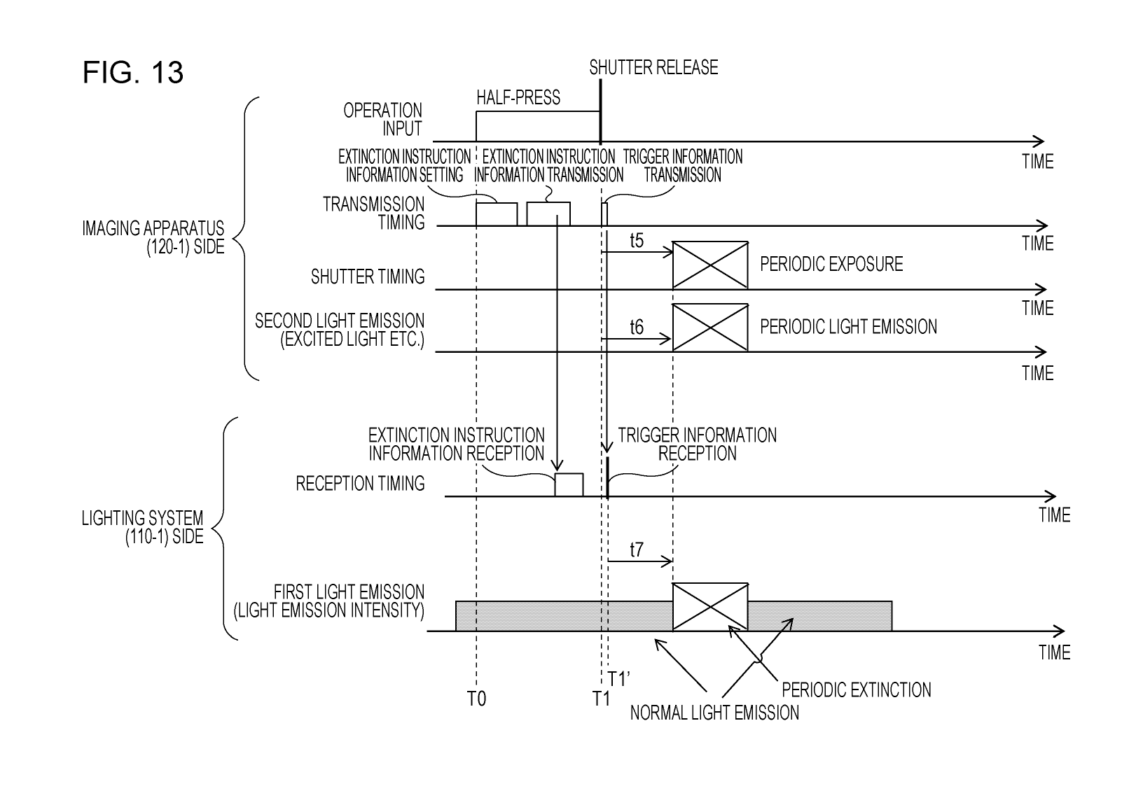

FIG. 13 is a timing chart illustrating an example processing procedure of the control method by the imaging system 100 illustrated in FIG. 1 according to the present exemplary embodiment.

In FIG. 13, the first to fourth stages from the top correspond to sequences of the imaging apparatus 120-1, and the fifth and sixth stages from the top correspond to sequences of the lighting system 110-1.

The sequence on the first stage from the top in FIG. 13 indicates the operation input from the user via the input device 227 of the imaging apparatus 120-1. In the example illustrated in FIG. 3, the half-press of the shutter button of the input device 227 is started at the time T0, and the indentation of the shutter button of the input device 227 is performed at the time T1.

The sequence on the second stage from the top in FIG. 13 indicates the transmission timing by the communication I/F 228 of the imaging apparatus 120-1 (the communication unit 123) to the lighting system 110-1.

First, when the half-press of the shutter button of the input device 227 occurs at the time T0, the CPU 221 of the imaging apparatus 120-1 identifies a timing in the periodic extinction period corresponding to a period during which the first illumination light 141 is periodically turned off and turn on from a surrounding situation or the like. Herein, in the example illustrated in FIG. 13, the lighting system 110-1 continuously emits the first illumination light 141 having a constant light intensity in the case of the normal light emission.

Subsequently, the CPU 221 of the imaging apparatus 120-1 sets the extinction instruction information (including information of the periodic extinction period) related to the identified timing in the periodic extinction period. Specifically, in the example illustrated in FIG. 13, the extinction instruction information is set which indicates that the first illumination light 141 is periodically turned off and turned on at a timing in a predetermined period after an elapse of a time t7 since the trigger information indicating that the indentation of the shutter button has been performed is received by the lighting system 110-1. Thereafter, the communication I/F 228 of the imaging apparatus 120-1 (the communication unit 123) transmits the extinction instruction information to the lighting system 110-1.

When the indentation of the shutter button of the input device 227 is performed, the CPU 221 of the imaging apparatus 120-1 detects this state. Then, the communication I/F 228 of the imaging apparatus 120-1 (the communication unit 123) transmits the trigger information indicating that the indentation of the shutter button has been performed to the lighting system 110-1.

The sequence on the third stage from the top in FIG. 13 indicates that the exposure is periodically performed (the electronic shutter is periodically opened) after an elapse of the time t5 since the trigger information is generated. Specifically, the CPU 221 of the imaging apparatus 120-1 sets the periodic exposure time corresponding to the time in which the periodic exposure is performed in accordance with the periodic extinction period of the first illumination light 141 instructed with respect to the lighting system 110-1.

The sequence on the fourth stage from the top in FIG. 13 indicates the second illumination light 142 is periodically emitted from the light emitting unit 226 after an elapse of the time t6 since the trigger information is generated. Specifically, the CPU 221 of the imaging apparatus 120-1 sets the periodic light emission time in which the periodic light emission of the second illumination light 142 is performed in accordance with the periodic extinction period of the first illumination light 141 instructed with respect to the lighting system 110-1.

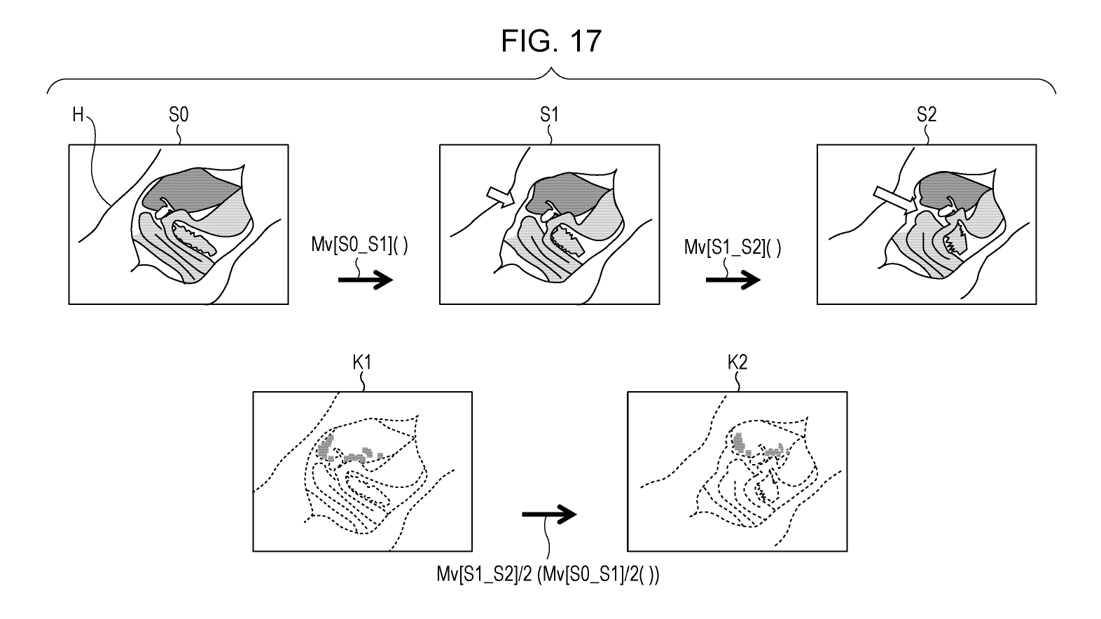

The sequence on the fifth stage from the top in FIG. 13 indicates the reception timing by the communication I/F 217 of the lighting system 110-1 (the communication unit 111). Specifically, in the example illustrated in FIG. 17, the reception timings of the extinction instruction information and the trigger information transmitted from the imaging apparatus 120-1 are indicated.

The sequence on the sixth stage from the top in FIG. 13 indicates timings of the normal light emission of the first illumination light 141 by the light emitting unit 215 of the lighting system 110-1 and the periodic extinction for periodically turning off and turning on the light, and the light emission intensity. Specifically, while the trigger timing T1′ set when the communication I/F 217 (the communication unit 111) receives the trigger information is used as the reference, the CPU 211 of the lighting system 110-1 performs the normal light emission until the time t7 elapses and performs the periodic extinction over a predetermined period after the elapse of the time t7. It is noted that, according to the exemplary embodiment, it is conceivable that t1≈t1′ is set.

Herein, as one of the characteristics of the present exemplary embodiment, in the above-described periodic extinction for periodically turning off and turning on the light, the length of the instantaneous extinction corresponding to the single extinction is suppressed to such an extent that the instantaneous extinction is not recognized as flickering or the like within a range of human visual responsiveness. Specifically, for example, the length of the instantaneous extinction is suppressed to such an extent that the operator O does not recognize the extinction of the first illumination light 141. The length of this instantaneous extinction is approximately 10 msec to 20 msec although differences exist between individuals.

In the descriptions using FIG. 13, it is supposed that the first illumination light 141 having the constant light intensity is continuously emitted in the case of the normal light emission.

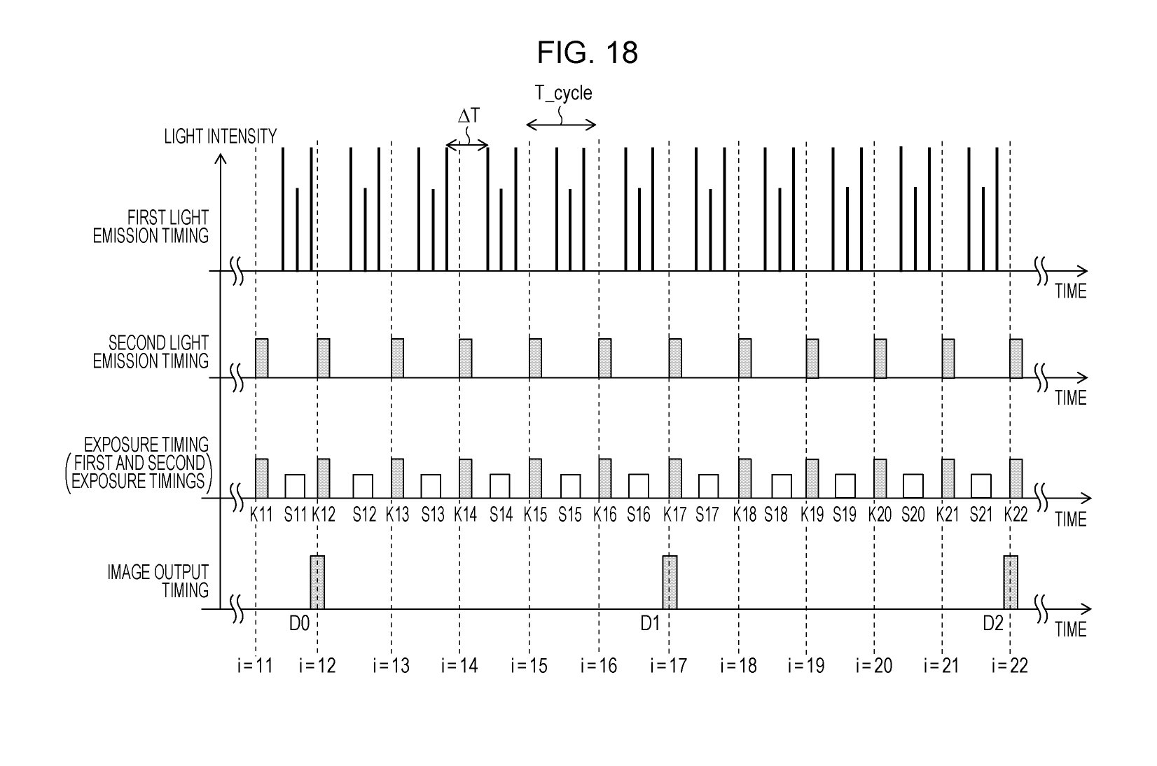

On the other hand, in actuality, the driving is performed in an impulse manner in a large number of the LED illuminations and the organic electroluminescence illuminations. In view of the above, from FIG. 18 and subsequent drawings, an example will be described in which the light that turns on by the impulse driving is supposed as the first illumination light 141.

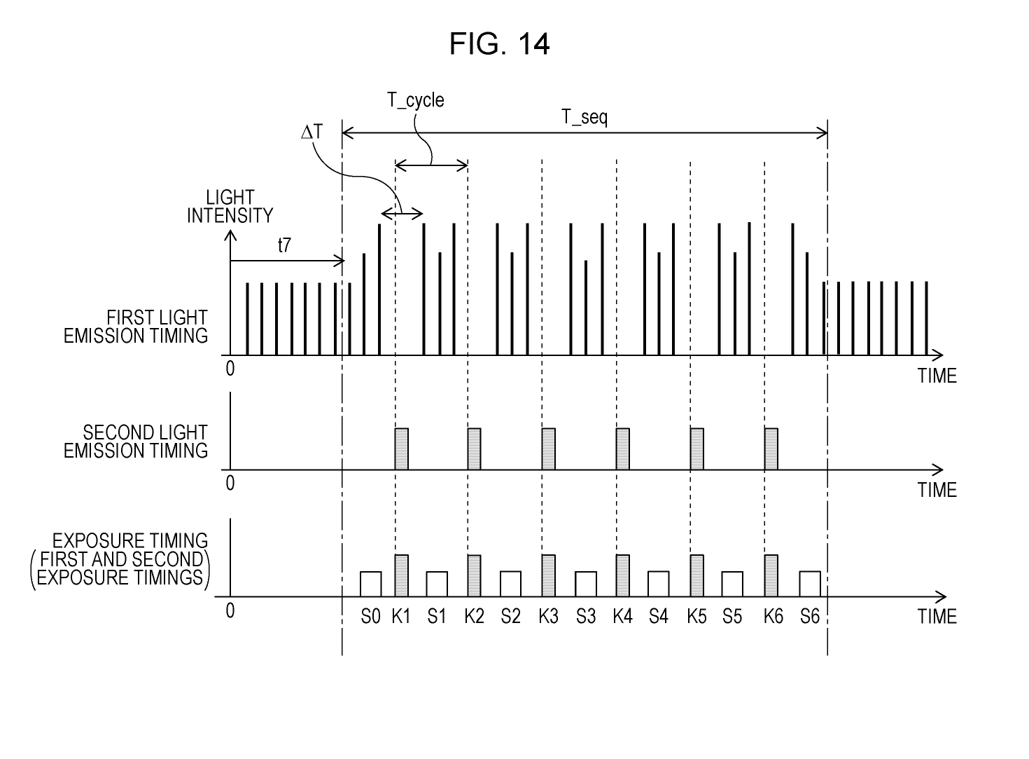

The frequency of the first illumination light 141 in the case of the impulse driving is approximately several hundreds of hertz to 1 kilohertz in general. It is noted that pulse trains in FIG. 14 and subsequent drawings are schematically represented, and intervals of the pulse trains in the drawings do not correctly represent the lighting frequencies.

FIG. 14 is a timing chart illustrating examples of the light emission timing of the first illumination light 141 in the normal light emission period and the periodic extinction period and the light emission timing and the exposure timing of the second illumination light 142 in the periodic extinction period.

It is noted that, in FIG. 14, a time (t1≈t1′) of the timing of the trigger information illustrated in FIG. 13 is set as a time 0.

The sequence on the first stage from the top in FIG. 14 indicates the light emission timing of the first illumination light 141 (the first light emission timing), the normal light emission is performed until the time t7 elapses, and the periodic extinction is performed over a predetermined period (T_seq) after the elapse of the time t7. In the example of FIG. 14, it is indicated that the instantaneous extinction having a length of a time ΔT is performed six times at a period (cycle) T_cycle within the predetermined time period (T_seq).

In a period immediately before and a period immediately after the instantaneous extinction, the CPU 211 of the lighting system 110-1 performs a control to carry out the irradiation of the first illumination light 141 having the light intensity higher than the normal light intensity of the illumination in the periodic extinction, so that the flickering or the like accompanied by the instantaneous extinction does not visually occur.

The sequence on the second stage from the top in FIG. 14 indicates the light emission timing of the second illumination light 142 (the second light emission timing) in the periodic extinction period (T_seq). Specifically, the light emission timing of the second illumination light 142 is set within the time of the instantaneous extinction of the first illumination light 141 (within the time ΔT).

The sequence on the third stage from the top in FIG. 14 indicates the exposure timing by the image pickup unit 225 of the imaging apparatus 120-1. The exposure is performed in the periodic extinction period (T_seq), and the first exposure performed in a period during which the first illumination light 141 is emitted (turned on) and the second exposure performed in a period during which the second illumination light 142 is emitted (turned on) are alternately carried out. Then, in order that each of the exposures can be calculated later, this is taken as an image (image data) for every exposure. That is, the first imaging by the first exposure is performed, and the second imaging by the second exposure is performed. Specifically, in the example illustrated in FIG. 14, a plurality of first images (illumination images) S0 to S6 obtained by performing the first imaging by the first exposure plural times and a plurality of second images (observation images) K1 to K6 obtained by performing the second imaging by the second exposure plural times are illustrated.

Exposure conditions appropriate to respective conditions are set for the first imaging by the first exposure and the second imaging by the second exposure.

Specifically, according to the present exemplary embodiment, a condition is set for the first exposure such that, for example, every exposure creates an image having a sufficient level. On the other hand, with regard to the second exposure, the single exposure is not sufficient, for example. Herein, a condition is set for the second exposure such that, for example, an image having a sufficient level is created by six exposures.

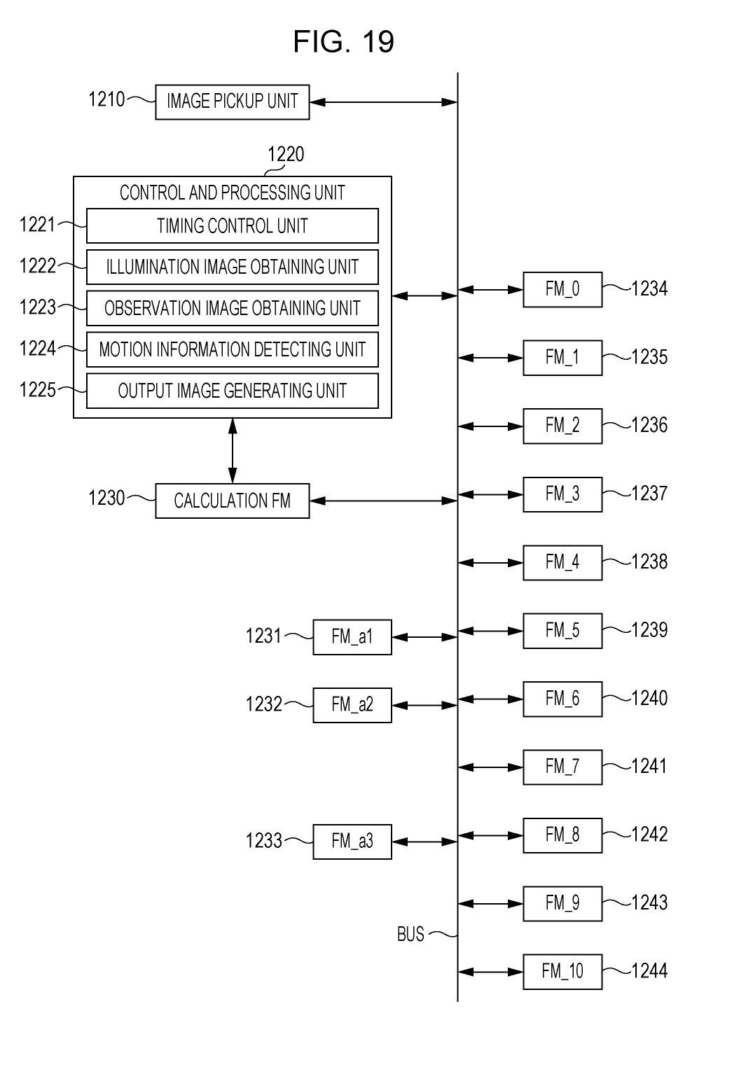

According to the present exemplary embodiment, the CPU 221 of the imaging apparatus 120-1 controls timings related to the first imaging, timings related to the second imaging, and the like. The CPU 221 of the imaging apparatus 120-1 which performs this timing control constitutes a timing control unit (a timing control unit 1221 illustrated in FIG. 19 which will be described below).

Specifically, as illustrated in FIG. 14, the CPU 221 of the imaging apparatus 120-1 performs a control such that the timing for emitting the first illumination light 141 related to the first imaging (the first light emission timing) and the timing for emitting the second illumination light 142 related to the second imaging (the second light emission timing) are not overlapped with each other. As illustrated in FIG. 14, the CPU 221 of the imaging apparatus 120-1 performs a control such that the timing for emitting the first illumination light 141 related to the first imaging (the first light emission timing) and the timing for the second imaging (the second exposure) are not overlapped with each other. As illustrated in FIG. 14, the CPU 221 of the imaging apparatus 120-1 controls the timing related to the first imaging and the timing related to the second imaging such that the first imaging (the first exposure) and the second imaging (the second exposure) are alternately carried out.

Subsequently, the CPU 221 of the imaging apparatus 120-1 obtains the plurality of first images obtained by performing the first imaging plural times (the illumination images S0 to S6 illustrated in FIG. 14) in the image pickup unit 225. The CPU 221 of the imaging apparatus 120-1 which performs the processing of obtaining the first images constitutes a first obtaining unit (an illumination image obtaining unit 1222 illustrated in FIG. 19 which will be described below).

The CPU 221 of the imaging apparatus 120-1 also obtains the plurality of second images (the observation images K1 to K6 illustrated in FIG. 14) obtained by performing the second imaging plural times in the image pickup unit 225. The CPU 221 of the imaging apparatus 120-1 which performs the processing of obtaining the second images constitutes a second obtaining unit (an observation image obtaining unit 1223 illustrated in FIG. 19 which will be described below).

Subsequently, the CPU 221 of the imaging apparatus 120-1 detects motion information related to a motion of the object H between the plurality of obtained first images (between the illumination images). The CPU 221 of the imaging apparatus 120-1 which performs the processing of detecting this motion information constitutes a motion information detecting unit (a motion information detecting unit 1224 illustrated in FIG. 19 which will be described below).

Subsequently, the CPU 221 of the imaging apparatus 120-1 performs first processing on the plurality of obtained second images on the basis of the detected motion information and performs second processing on the plurality of second images on which the first processing has been performed to generate a single output image. The CPU 221 of the imaging apparatus 120-1 which performs the processing of generating this output image constitutes an output image generating unit (an output image generating unit 1225 illustrated in FIG. 19 which will be described below).

Next, the detection processing for the motion information by the CPU 221 of the imaging apparatus 120-1 will be described.

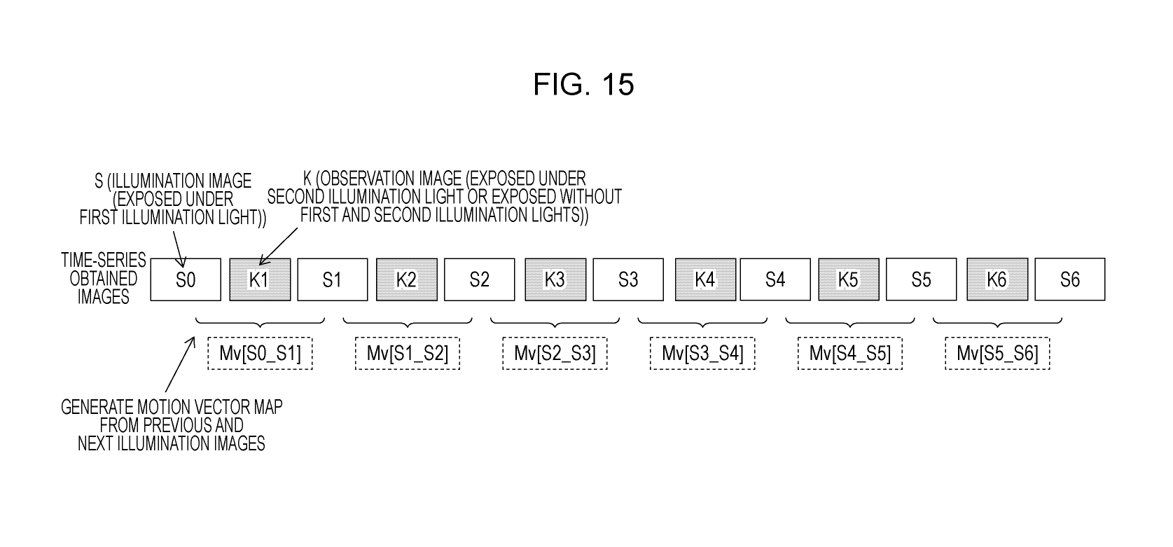

Herein, a motion vector map determined by a transition from an image I to an image J is defined as Mv[I_J]. Hereinafter, descriptions will be given by using FIG. 15.

FIG. 15 is an explanatory diagram for describing the detection processing for the motion information by the imaging apparatus 120-1 according to the first exemplary embodiment of the present disclosure.

Herein, FIG. 15 illustrates images obtained in a time-series manner by the control of the exposure timings for the first exposure and the second exposure illustrated in FIG. 14. Specifically, as the time-series obtained images, FIG. 15 illustrates an example in which the illumination image S0, the observation image K1, the illumination image S1, the observation image K2, the illumination image S2, the observation image K3, the illumination image S3, the observation image K4, the illumination image S4, the observation image K5, the illumination image S5, the observation image K6, and the illumination image S6 are obtained in the time-series manner. It is noted that, in the above-described explanations, the observation image corresponding to the second image obtained by way of the second imaging by the second exposure is imaged in a state in which the second illumination light 142 is emitted, but the present disclosure is not limited to this configuration. For example, as illustrated in FIG. 15, the observation image corresponding to the second image obtained by way of the second imaging by the second exposure may be imaged in a state in which none of the first illumination light 141 and the second illumination light 142 are emitted.

As illustrated in FIG. 15, a motion vector map determined by the combination of the illumination image S0 and the illumination image S1 obtained by way of the first imaging by the first exposure is defined as Mv[S0_S1]. Similarly, a motion vector map determined by the combination of the illumination image S1 and the illumination image S2 is defined as Mv[S1_S2], and the same applied to the combination of the illumination image S2 and the illumination image S3 and the subsequent combinations.

Herein, processing of applying the motion vector map Mv[I_J] determined by the images I and J to an image X and generating an image Y (that is, motion compensation is performed) is represented by the following Expression (2).

Y=Mv[I_{—}J](x) (2)

Next, descriptions will be given by using images of specific pictures.

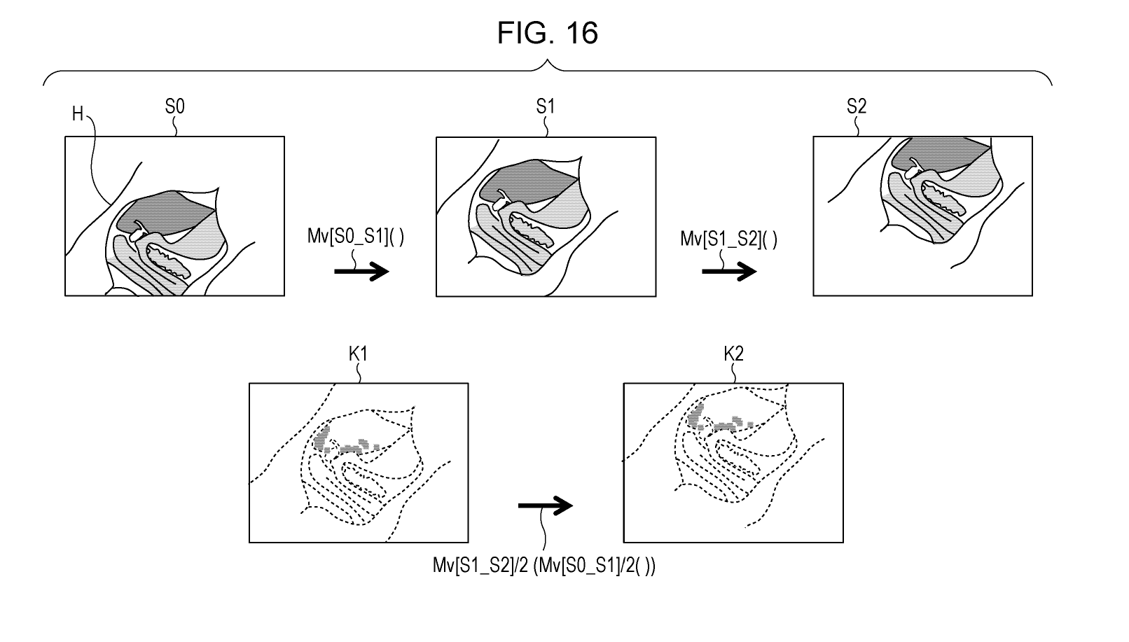

FIG. 16 is an explanatory diagram for describing the detection processing for the motion information by the imaging apparatus 120-1 according to the first exemplary embodiment of the present disclosure.

According to the present exemplary embodiment, a case is supposed where the first imaging (the first exposure) for obtaining the illumination image S and the second imaging (the second exposure) for obtaining the observation image K are imaged while being respectively shifted by just a half of the period in the same period.

As illustrated in FIG. 16, the illumination image S0, the observation image K1, the illumination image S1, the observation image K2, and the illumination image S2 are imaged in a time-series manner. Herein, in the example illustrated in FIG. 16, contents of the images correspond to an instance where the object H existing across the entire image is gradually moved in an upper right direction over time. This state occurs, for example, in a case where a position of the object H is changed, a case where the imaging apparatus 120-1 trembles so that the position or an imaging direction is changed, or the like. To represent the state in which the object H is moved in the upper right direction, the motion vector maps Mv[S0_S1] and Mv[S1_S2] between the respective illumination images are obtained. This corresponds to the motion detection of the object H based on the illumination image S. Herein, in the case illustrated in FIG. 16, for example, information related to a translational movement or a rotational movement across the entire image of the object H the plurality of illumination images is detected as the motion information.

Herein, a transform from the illumination image S0 to the illumination image S1 is represented by a function Mv[S0_S1]( ). Similarly, a transform from the illumination image S1 to the illumination image S2 is represented by a function Mv[S1_S2] ( ). Herein, according to the present exemplary embodiment, as described above, the exposure timing of the observation image K1 is supposed to be at the midpoint between the exposure timing of the illumination image S0 and the exposure timing of the illumination image S1. Similarly, the exposure timing of the observation image K2 is supposed to be at the midpoint between the exposure timing of the illumination image S1 and the exposure timing of the illumination image S2.

Herein, although the exposure conditions are different from each other, the illumination image S and the observation image K are obtained by imaging the same object H from the same direction at the same field angle. For this reason, the illumination image S and the observation image K are meant to have a series of motions on the same line while a time difference is taken into account.

As described above, the exposure timing of the observation image K1 is at the midpoint between the exposure timing of the illumination image S0 and the exposure timing of the illumination image S1. For this reason, the motion of the object H from the observation image K1 to the observation image K2 is substantially the same as a motion obtained by overlapping the transforms with each other in which the motions of the object H of the previous and next illumination images S are respectively halved.

That is, the transform from the observation image K1 to the observation image K2 can be represented as follows.

### Mv[S1_S2]/2 (Mv[S0_S1]/2 ( ))

Therefore, when an image obtained by estimating an image at a time point time of the observation image K2 from the observation image K1 (image obtained by moving the observation image K1 to the time point of the observation image K2 on the basis of the motion information) is set as Kv1_2, this can be represented by the following Expression (3).

Kv1_{—}2=Mv[S1_{—}S2]/2(Mv[S0_{—}S1]/2(K1) (3)

By generalizing this, for example, in a case where the exposure timing of the observation image K is a timing having a ratio of a:b (a+b=1, a>0, b>0) with respect to the period of the exposure timing of the illumination image S, Expression (3) can be rewritten into the following Expression (4).

Kv1_{—}2=Mv[S1_{—}S2]*a(Mv[S0_{—}S1]*b(K1) (4)

According to the present exemplary embodiment, Expression (3) corresponds to a case where a=0.5 and b=0.5 are set in Expression (4).

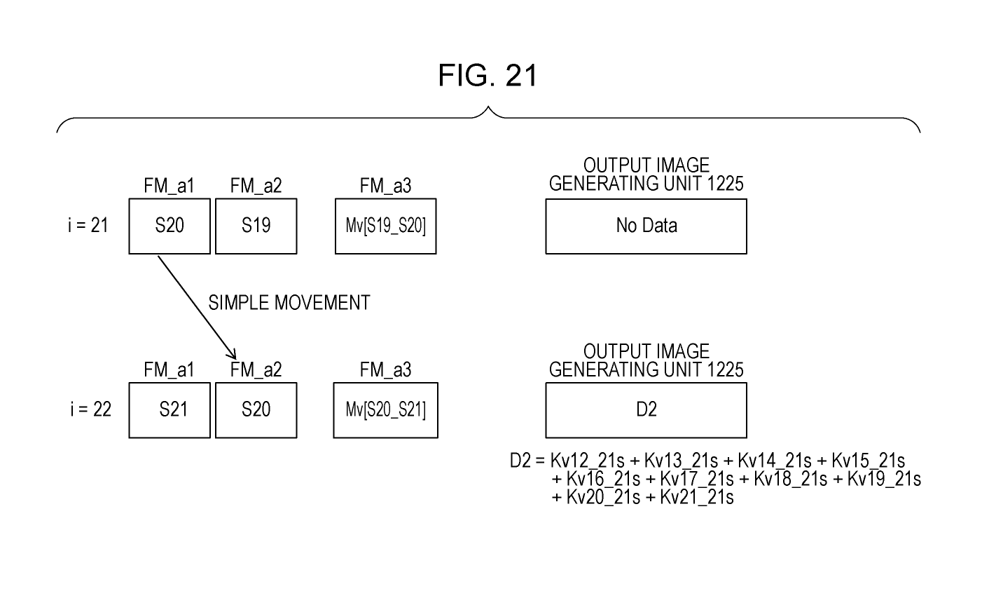

FIG. 17 is an explanatory diagram for describing the detection processing for the motion information by the imaging apparatus 120-1 according to the first exemplary embodiment of the present disclosure. FIG. 17 indicates a case where the motion of the object H varies for each image region, that is, a case where the object H is partially deformed instead of a case where the motion of the object H exists across the entire image (the same across all the regions of the image) as illustrated in FIG. 16. For example, in an example illustrated in FIG. 21, deformation or the like of the object H which occurs when a part of the object H indicated by an empty arrow is pressed is illustrated. Herein, in the example illustrated in FIG. 21, information related to the translational movement or the rotational movement for each image region of the object H between the plurality of illumination images is detected as the motion information, for example.

Even in a case where the partial movement occurs in the object H in the above-described manner, Expression (3) and Expression (4) described above are established similarly as described in FIG. 16.

Herein, with the same rule as the image Kv1_2 described above, with regard to each of the observation images K1, K2, K3, K4, and K5, images obtained by estimating images at the time point of the observation image K6 (images obtained by moving the observation images K1, K2, K3, K4, and K5 to the time point of the observation image K6 on the basis of the motion information) are respectively set as images Kv1_6, Kv2_6, Kv3_6, Kv4_6, and Kv5_6.

The images Kv1_6, Kv2_6, Kv3_6, Kv4_6, and Kv5_6 can be respectively represented by the following Expressions (5-1) to (5-5) on the basis of the above-described concept.

Kv1_{—}6=Mv[S5_{—}S6]/2(Mv[S4_{—}S5](Mv[S3_{—}S4](Mv[S2_{—}S3](Mv[S1_{—}S2](Mv[S0_{—}S1]/2(K1)))))) (5-1)

Kv2_{—}6=Mv[S5_{—}S6]/2(Mv[S4_{—}S5](Mv[S3_{—}S4](Mv[S2_{—}S3](Mv[S1_{—}S2]/2(K2))))) (5-2)

Kv3_{—}6=Mv[S5_{—}S6]/2(Mv[S4_{—}S5](Mv[S3_{—}S4](Mv[S2_{—}S3]/2(K3)))) (5-3)

Kv4_{—}6=Mv[S5_{—}S6]/2(Mv[S4_{—}S5](Mv[S3_{—}S4]/2(K4))) (5-4)

Kv5_{—}6=Mv[S5_{—}S6]/2(Mv[S4_{—}S5]/2(K5)) (5-5)

Kv6_{—}6=K6 (5-6)

The processing performed in Expressions (5-1) to (5-5) is so-called positioning processing. Since Kv6_6 becomes the observation image K6, Expression (5-6) is obviously established. That is, the processing represented in Expressions (5-1) to (5-6) are equivalent to the first processing performed on the plurality of observation images (the second images) on the basis of the detected motion information. With this first processing, the positioning processing by correcting the translational movement or the rotational movement across the entire image of the object H or the positioning processing by correcting the translational movement or the rotational movement for each image region of the object H is performed.

Furthermore, addition processing is performed on the images Kv1_6 to Kv6_6 illustrated in Expressions (5-1) to (5-6) as in the following Expression (5) to generate one output image K_sum. In a case where a situation occurs in which a pixel value of the output image K_sum is saturated or the like, averaging processing is performed on the images Kv1_6 to Kv6_6 illustrated in Expressions (5-1) to (5-6) as in the following Expression (7) as needed to generate one output image K_ave. The processing indicated by Expression (6) or Expression (7) is equivalent to the second processing performed on the plurality of observation images on which the above-described first processing has been performed when the single output image is generated.

K_{—}sum=Kv1_{—}6+Kv2_{—}6+Kv3_{—}6+Kv4_{—}6+Kv5_{—}6+Kv6_{—}6 (6)

K_{—}ave=(Kv1_{—}6+Kv2_{—}6+Kv3_{—}6+Kv4_{—}6+Kv5_{—}6+Kv6_{—}6)/6 (7)

Herein, the output image K_sum or the like indicated in Expression (6) is by no means inferior in terms of luminance or S/N and also by far superior in terms of moving image blurring or irregular deviation if this is compared with the single imaging by the exposure for the total exposure time of the observation images K1 to K6 while the object H is fixed.

According to the present exemplary embodiment, it is possible to obtain an appropriate object image (output image) of the object H in motion. In addition, according to the present exemplary embodiment, since the control is performed such that the timing for emitting the first illumination light 141 related to the first imaging is not overlapped with the timing for emitting the second illumination light 142 related to the second imaging (or the second imaging timing), it is possible to obtain the appropriate object image (output image) of the object H in motion while the work is carried out by the operator O who performs the work on the object H in motion. Accordingly, it is possible to perform the pathological diagnosis based on the object image (output image) in the actual time during the operation of the object H, for example, and it is possible to carry out the appropriate operation.

### Sixth Exemplary Embodiment

Next, a sixth exemplary embodiment of the present disclosure will be described.

According to the sixth exemplary embodiment, instead of generating the single output image from the exposures performed the particular number of times as in the processing described in the above-described fifth exemplary embodiment, the similar processing is sequentially continuously performed with respect to the images obtained by the sequential exposures.

The imaging system according to the sixth exemplary embodiment is similar, for example, to the imaging system 100 according to the first exemplary embodiment illustrated in FIG. 1. In addition, according to the present exemplary embodiment, moving images are output as the output of the imaging apparatus.

FIG. 18 is a timing chart illustrating examples of the light emission timing of the first illumination light 141, the light emission timing of the second illumination light 142, the exposure timing, and the image output timing according to the sixth exemplary embodiment of the present disclosure.

According to the sixth exemplary embodiment too, similarly as in the fifth exemplary embodiment, the first light emission by the first illumination light 141 occurs at a predetermined period, and the second light emission by the second illumination light 142 is performed in the extinction period of this first light emission. That is, the first imaging based on the first exposure is performed in the first light emission period, and the second imaging based on the second exposure is performed in the second light emission period.

Hereinafter, the processing procedure of the control method by the imaging apparatus according to the present exemplary embodiment will be described.

According to the present exemplary embodiment, with regard to second images imaged at a predetermined frame rate, an output image is generated by positioning frames of m2 pieces of previous images for every m1 frames. Herein, m1 and m2 are integers. That is, when a frame rate of the second images is set as F0, a frame rate of the output image is F0/m1, and the output image is obtained by making a reference and positioning the frames of the m2 pieces of previous images. According to the present exemplary embodiment, the frame rate F0 is not particularly determined, but a case where m1=5 and m2=10 are set will be hereinafter described as an example.

The sequence on the first stage from the top in FIG. 18 indicates the light emission timing of the first illumination light 141 (the first light emission timing), and the instantaneous extinction having the length of the time ΔT in the period T_cycle is performed.

The sequence on the second stage from the top in FIG. 18 indicates the light emission timing of the second illumination light 142 (the second light emission timing), and the second light emission by the second illumination light 142 is performed at the period T_cycle within the time period of the instantaneous extinction of the first illumination light 141.

The sequence on the third stage from the top in FIG. 18 indicates the exposure timing by the image pickup unit 225 of the imaging apparatus according to the present exemplary embodiment. With regard to the exposure, the first exposure performed while the first illumination light 141 is emitted (turned on) and the second exposure performed while the second illumination light 142 is emitted (turned on) are alternately carried out. Then, in order that each of the exposures can be calculated later, this is taken as an image (image data) for every exposure. That is, the first imaging by the first exposure is performed, and the second imaging by the second exposure is performed. Specifically, in the example illustrated in FIG. 18, a plurality of first images (illumination images) S11 to S21 obtained by performing the first imaging by the first exposure plural times and a plurality of second images (observation images) K11 to K22 obtained by performing the second imaging by the second exposure plural times are illustrated.

The sequence on the fourth stage from the top in FIG. 18 indicates output timings of output images D0 to D2. As illustrated in FIG. 18, the single output image D is output for every five frames of the input images (displayed on the third stage).

Hereinafter, an example of processing of generating the output image D2 illustrated in FIG. 18 will be described.

The output image D2 is obtained in the following manner. That is, images obtained by virtually moving the observation images K12, K13, K14, K15, K16, K17, K18, K19, K20, and K21 illustrated in FIG. 18 to an imaging timing of the illumination image S21 on the basis of the motion information obtained from the illumination image S are added to one another or averaged.

Herein, the images obtained by moving the observation images K12 to K21 as described above to the imaging timing of the illumination image S21 are represented as follows.

K12_21s, K13_21s, K14_21s, K15_21s, K16_21s, K17_21s, K18_21s, K19_21s, K20_21s, K21_21s

Then, the total sum or the average of these images corresponds to the output image D2.

Herein, a process in which the first observation images K12 is transformed into K12_21s can be decomposed as follows.

K12→K12_12s→K12s_13s→K13s_14s→ . . . →K19s_20s→K20s_21s

Herein, only the first transform is performed in a manner that each component of the motion vector map determined by the illumination images S having the adjacent sequential orders is halved. After that, all the transforms are based on the motion vector map determined by the mutual illumination images S having the adjacent sequential orders. In any one of the observation images K12 to K21, the transform is based on the half of the motion vector map only for the first time immediately after the imaging as described above, and thereafter, the transform is based on the above-described motion vector map.

Next, a function configuration of the imaging apparatus according to the present exemplary embodiment will be described.

FIG. 19 illustrates an example functional configuration of the imaging apparatus according to the sixth exemplary embodiment of the present disclosure. Herein, FIG. 19 illustrates only a part according to the present disclosure among the functional configurations according to the present exemplary embodiment.

As illustrated in FIG. 19, the imaging apparatus according to the present exemplary embodiment is constituted by including an image pickup unit 1210, a control and processing unit 1220, a calculating frame memory (calculating FM) 1230, and various frame memories (FMs) 1231 to 1244.

The image pickup unit 1210 is configured to pick up an image based on the light 143 from the object H by performing the imaging of the object H. The image pickup unit 1210 is constituted, for example, by the image pickup unit 225 illustrated in FIG. 2B or the like (the image pickup unit 121 illustrated in FIG. 1 or the like).

The control and processing unit 1220 controls the operation by the imaging apparatus according to the present exemplary embodiment in an overall manner and also performs various processings. The control and processing unit 1220 is configured, for example, while the CPU 221 illustrated in FIG. 2B or the like executes the program stored in the external memory 224.

Specifically, the control and processing unit 1220 includes the timing control unit 1221, the illumination image obtaining unit 1222, the observation image obtaining unit 1223, the motion information detecting unit 1224, and the output image generating unit 1225.

The timing control unit 1221 controls the timing related to the first imaging performed in the image pickup unit 1210 and the timing related to the second imaging performed in the image pickup unit 1210.

The illumination image obtaining unit 1222 obtains the plurality of illumination images (S11 to S21 in FIG. 18) corresponding to the plurality of first images obtained by performing the first imaging plural times in the image pickup unit 1210 from the image pickup unit 1210.

The observation image obtaining unit 1223 obtains the plurality of observation images (K11 to K22 in FIG. 18) corresponding to the plurality of second images obtained by performing the second imaging plural times in the image pickup unit 1210 from the image pickup unit 1210.

The motion information detecting unit 1224 detects the motion information related to the motion of the object H between the plurality of illumination images obtained in the illumination image obtaining unit 1222.

First, the output image generating unit 1225 performs first processing on the plurality of observation images obtained by the observation image obtaining unit 1223 on the basis of the motion information detected by the motion information detecting unit 1224. Herein, the first processing corresponds to the positioning processing by correcting the translational movement or the rotational movement across the entire image of the object H with respect to the plurality of observation images or the positioning processing by correcting the translational movement or the rotational movement for each image region of the object H with respect to the plurality of observation images. Subsequently, the output image generating unit 1225 performs second processing on the plurality of observation images on which the first processing has been performed to generate a single output image. Herein, the second processing corresponds to addition processing of the images or the averaging processing of the images with respect to the plurality of observation images on which the first processing has been applied.

The control and processing unit 1220 performs calculation processing while information is temporarily stored in the calculating FM 1230 via the bus and also transfers a result of the calculation processing to each block via the bus.

The calculating FM 1230 stores the information such as the result of the calculation processing by the control and processing unit 1220 and is used when the control and processing unit 1220 performs the calculation processing.

The FM_a1 (1231) to the FM_a3 (1233) and the FM_0 (1234) to the FM_10 (1244) are so-called frame memories.

Next, the calculation processing using the various frame memories by the control and processing unit 1220 will be described.

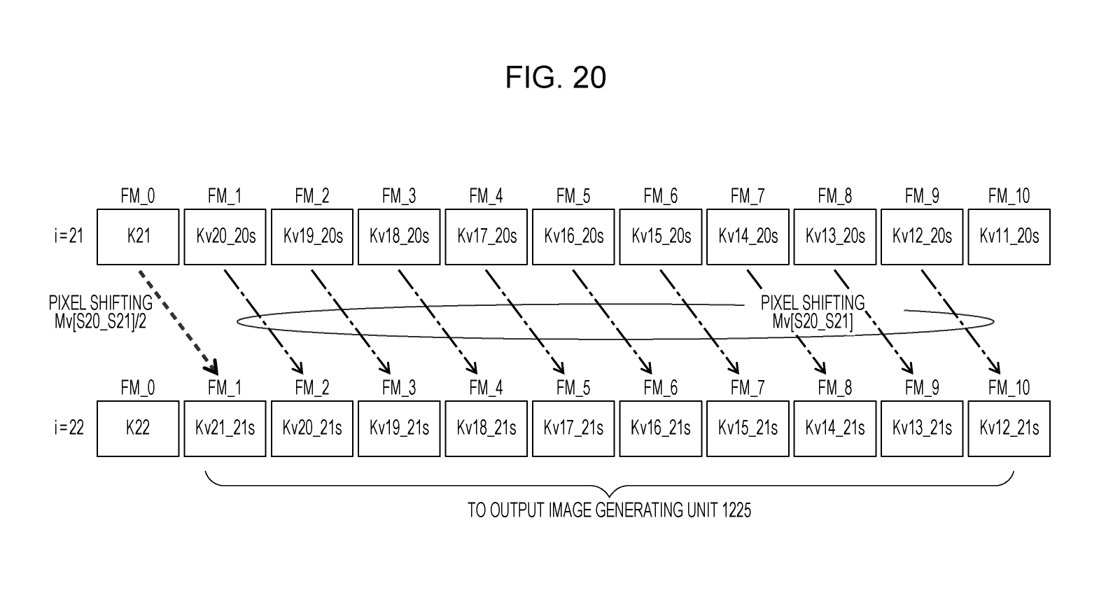

FIG. 20 is an explanatory diagram for describing the calculation processing by using the respective frame memories by the control and processing unit illustrated in FIG. 19 according to the sixth exemplary embodiment of the present disclosure. Specifically, FIG. 20 illustrates a movement of the data between the respective frame memories for realizing the calculation processing by the control and processing unit 1220.

An upper stage in FIG. 20 indicates contents of the respective frame memories immediately after the observation image K21 is input (at a time point of i=21 in FIG. 18). A lower stage in FIG. 20 indicates contents of the respective frame memories immediately after the observation image K22 is input (at a time point of i=22 in FIGS. 11A to 11E).

An image stored in the frame memory FM_0 in FIG. 20 is applied with the transform where the motion amount of the motion vector map determined by the illumination images S is halved each time i is incremented and moved to the frame memory FM_1. Herein, at the time of i=21→i=22, this transform (former transform) is represented by Y=Mv[S20_S21]/2 (X).

Images stored in the frame memories FM_1 to FM_9 are applied with the transform based on the motion vector map determined by the illumination images S each time i is incremented and moved to the frame memory FM on the immediate right. Herein, at the time of i=21→i=22, this transform (latter transform) is represented by Y=Mv[S20_S21] (X).

In FIG. 20, the former transform and movement are represented by a broken line arrow, and the latter transform and movement are represented by dashed-dotted line arrows.

According to the present exemplary embodiment, the output image is output at an interval of once for every five frames of input frames (imaged frames). That is, within a range illustrated in FIG. 18, the output images D1, D2, and D3 are respectively output at timings of i=12, 17, and 22.

Herein, for example, the output image D2 is obtained while the images obtained by transforming the observation images K12 to K21 to the timing of the illumination image S21 are added to one another.

FIG. 21 is an explanatory diagram for describing the calculation processing by using the respective frame memories by the control and processing unit illustrated in FIG. 19 according to the sixth exemplary embodiment of the present disclosure. Specifically, FIG. 21 illustrates contents of the images stored in the respective frame memories FM_a1, FM_a2, and FM_a3 and the output image generated by the output image generating unit 1225.

At the time point of i=21, the illumination images S20 and S19 are respectively stored in the frame memories FM_a1 and FM_a2, and the motion vector map Mv[S19_S20] calculated on the basis of these illumination images is stored in the frame memory FM_a3.

At the time point of i=22, the illumination images S21 and S20 are respectively stored in the frame memories FM_a1 and FM_a2, and the motion vector map Mv[S20_S21] calculated on the basis of these illumination images is stored in the frame memory FM_a3.

The images stored in the frame memories FM_1 to FM_9 at the time point of i=21 are transformed by the motion vector map Mv[S20_S21] at the time point of i=22 and moved to the frame memories FM 2 to FM_10.

In addition, the image stored in the frame memory FM_0 at the time point of i=21 is transformed by the motion vector map Mv[S20_S21]/2 at the time point of i=22 and stored in the frame memory FM_1.

Furthermore, since the time point of i=22 is the timing for outputting the output image D2, all the images stored in the frame memories FM_1 to FM_10 are added to one another (may be averaged when needed) to generate and output the output image D2.

In this manner, first, the output image generating unit 1225 sequentially performing positioning of the observation images K imaged at the frame rate F0 on the basis of the motion information obtained by the illumination images S up to m2 times. On that basis, the output image generating unit 1225 adds (averages) the m2 pieces of frames to one another for every m1 pieces of frames (that is, at the frame rate of F0/m1) to generate the output image D. With this configuration, the images imaged at the frame rate F0 are output as moving images where the frame rate is converted to 1/m1.

The respective frames of the output moving images are images obtained by positioning and adding the immediately preceding m2 pieces of input images or the like. Accordingly, the output images hardly has dynamic deterioration such as motion blurring or multiply-layered images, and the S/N improvement and level improvement are achieved, so that the low luminance imaging or the like of the moving object H is realized.

### Other Embodiments

Embodiments of the present disclosure can also be realized by a computer of a system or apparatus that reads out and executes computer executable instructions recorded on a storage medium (e.g., non-transitory computer-readable storage medium) to perform the functions of one or more of the above-described embodiment(s) of the present disclosure, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present inventions have been described with reference to exemplary embodiments, it is to be understood that the inventions are not limited to the disclosed exemplary embodiments. The scope of the following claims is to be accorded the broadest interpretation so as to encompass all such modifications and equivalent structures and functions.

This application claims the benefit of Japanese Patent Application No. 2014-178481, filed Sep. 2, 2014, and Japanese Patent Application No. 2014-178483, filed Sep. 2, 2014, which applications are hereby incorporated by reference herein in their entireties.

What is claimed is:

1. An imaging apparatus that performs imaging of an object in an environment irradiated with illumination light by a lighting system, the imaging apparatus comprising:
an input unit configured to input an instruction for performing the imaging;
an identification unit configured to identify a timing in an extinction period of the illumination light at or during which an operator who performs a work on the object does not recognize an extinction of the illumination light corresponding to a timing after the instruction for performing the imaging is input from the input unit; and
an image pickup unit configured to perform the imaging of the object to pick up an object image at the timing in the extinction period identified by the identification unit.

2. The imaging apparatus according to claim 1, further comprising:
a communication unit configured to perform a communication with the lighting system,
wherein the communication unit transmits extinction instruction information related to the timing in the extinction period identified by the identification unit to the lighting system.

3. The imaging apparatus according to claim 1, further comprising:
a light receiving unit configured to receive the illumination light,
wherein the identification unit identifies the timing in the extinction period on a basis of a waveform of the illumination light received by the light receiving unit.

4. The imaging apparatus according to claim 1, further comprising:
a light emitting unit configured to emit second illumination light to the object which is different light from the illumination light in accordance with a purpose of the imaging when the image pickup unit performs the imaging of the object.

5. The imaging apparatus according to claim 4, wherein the second illumination light is light that excites a predetermined fluorescent material.

6. The imaging apparatus according to claim 1, further comprising:
a setting unit configured to set a shutter time corresponding to a time in which a shutter is opened in accordance with the extinction period.

7. A lighting system that is communicable with an imaging apparatus configured to perform imaging of an object and irradiates the object with illumination light having a first light intensity, the lighting system comprising:
a communication unit configured to perform a communication with the imaging apparatus and receive extinction instruction information related to a timing in an extinction period of the illumination light from the imaging apparatus; and
a control unit configured to control an extinction of the illumination light on a basis of the extinction instruction information and perform a control to emit illumination light having a second light intensity higher than the first light intensity in at least one of a period immediately before and a period immediately after the extinction period of the illumination light on a basis of a predetermined rule.

8. The lighting system according to claim 7, wherein the predetermined rule is a rule in which a change ratio in the extinction period of a value obtained by correcting a light intensity of the illumination light by a time integration characteristic based on a visual sensitivity characteristic becomes lower than the illumination light that keeps the first light intensity.

9. A control method for an imaging apparatus that performs imaging of an object in an environment irradiated with illumination light by a lighting system, the control method comprising:
inputting an instruction for performing the imaging from an input unit of the imaging apparatus;
identifying a timing in an extinction period of the illumination light at or during which an operator who performs a work on the object does not recognize an extinction of the illumination light corresponding to a timing after the instruction for performing the imaging is input from the input unit, and
performing the imaging of the object to pick up an object image at the timing in the identified extinction period.

10. A control method for a lighting system that is communicable with an imaging apparatus configured to perform imaging of an object and irradiates the object with illumination light having a first light intensity, the control method comprising:
performing a communication with the imaging apparatus and receiving extinction instruction information related to a timing in an extinction period of the illumination light from the imaging apparatus; and
controlling an extinction of the illumination light on a basis of the extinction instruction information and performing a control to emit illumination light having a second light intensity higher than the first light intensity in at least one of a period immediately before and a period immediately after the extinction period of the illumination light on a basis of a predetermined rule.

11. An imaging apparatus comprising:
an image pickup unit configured to perform imaging of an object and obtain an image;
a timing control unit configured to control a timing related to a first imaging performed by the image pickup unit in a state in which the object is irradiated with a first illumination light and a timing related to a second imaging performed by the image pickup unit in a state in which the object is irradiated with a second illumination light that is different from the first illumination light or a state in which the object is irradiated with none of the first illumination light and the second illumination light;
a first obtaining unit configured to obtain a plurality of first images by performing the first imaging a plurality of times by the image pickup unit;
a second obtaining unit configured to obtain a plurality of second images by performing the second imaging a plurality of times by the image pickup unit;
a detection unit configured to detect motion information related to a motion of the object between the plurality of the first images obtained by the first obtaining unit; and
a generation unit configured to perform a first processing on the plurality of the second images by the second obtaining unit on a basis of the motion information detected by the detection unit and perform a second processing on the plurality of the second images on which the first processing has been performed to generate one output image.

12. The imaging apparatus according to claim 11, wherein the timing control unit performs a control in a manner that an illumination timing of the first illumination light related to the first imaging is not overlapped with an illumination timing of the second illumination light related to the second imaging.

13. The imaging apparatus according to claim 11, wherein the timing control unit performs a control in a manner that an illumination timing of the first illumination light related to the first imaging is not overlapped with a timing of the second imaging.

14. The imaging apparatus according to claim 11, wherein the timing control unit controls the timing related to the first imaging and the timing related to the second imaging in a manner that the first imaging and the second imaging are alternately performed.

15. The imaging apparatus according to claim 11, wherein the detection unit detects information related to a translational movement or a rotational movement across an entire image of the object between the plurality of the first images obtained by the first obtaining unit as the motion information.

16. The imaging apparatus according to claim 11, wherein the detection unit detects information related to a translational movement or a rotational movement for each image region of the object between the plurality of the first images obtained by the first obtaining unit as the motion information.

17. The imaging apparatus according to claim 15, wherein the generation unit performs processing of correcting the translational movement or the rotational movement across the entire image and positioning with respect to the plurality of the second images obtained by the second obtaining unit as the first processing.

18. The imaging apparatus according to claim 16, wherein the generation unit performs processing of correcting the translational movement or the rotational movement for each image region and positioning with respect to the plurality of the second images obtained by the second obtaining unit as the first processing.

19. The imaging apparatus according to claim 11, wherein the generation unit performs image addition processing or image averaging processing as the second processing on the plurality of the second images on which the first processing has been performed and generates the output image.

20. A control method for an imaging apparatus provided with an image pickup unit configured to perform imaging of an object and obtain an image, the control method comprising:
controlling a timing related to a first imaging performed by the image pickup unit in a state in which the object is irradiated with a first illumination light and a timing related to a second imaging performed by the image pickup unit in a state in which the object is irradiated with a second illumination light that is different from the first illumination light or a state in which the object is irradiated with none of the first illumination light and the second illumination light;
obtaining a plurality of first images by performing the first imaging a plurality of times by the image pickup unit;
obtaining a plurality of second images by performing the second imaging a plurality of times by the image pickup unit;
detecting motion information related to a motion of the object between the plurality of the obtained first images; and
performing a first processing on the plurality of the obtained second images on a basis of the detected motion information and performing a second processing on the plurality of the obtained second images on which the first processing has been performed to generate one output image.
